(19) United States
(12) Patent Application Publication
(10) Pub. No.: US 2021/0238229 A1
(43) Pub. Date: Aug. 5, 2021

(54) PEPTIDE FOR DISEASE TREATMENT

(71) Applicant: UNIVERSITY COLLEGE CORK - NATIONAL UNIVERSITY OF IRELAND, CORK, Cork (IE)

(72) Inventor: Thomas WALTHER, Cork (IE)

(73) Assignee: UNIVERSITY COLLEGE CORK - NATIONAL UNIVERSITY OF IRELAND, CORK, Cork (IE)

(21) Appl. No.: 17/253,914

(22) PCT Filed: Jun. 14, 2019
(86) PCT No.: PCT/EP2019/065776

§ 371 Date: Dec. 18, 2020

(30) Foreign Application Priority Data
Jun. 14, 2018 (EP) 18177878.8

(51) Int. Cl. C07K 7/14; C07K 7/06

(52) U.S. Cl. CPC C07K 7/14; A61K 38/00; C07K 7/06

(57) ABSTRACT

A peptide comprises an amino acid sequence of SEQUENCE ID NO. 1, in which the sequence has 1, 2 or 3 amino acid changes. The peptide has utility in treatment of cardiovascular disease and neurodegenerative disease.

### FIELD OF THE INVENTION

The current invention relates to cardiovascular disease treatment, neurodegenerative disease treatment and treatment of diseases with compromised barrier function, and a suitable pharmaceutical composition(s) for said treatments. The current invention also relates to the treatment of pain, in particular chronic pain, and a suitable pharmaceutical composition(s) for said treatment. The invention also relates to cancer treatment and suitable compositions for said treatment.

### BACKGROUND TO THE INVENTION

Cardiovascular disease (CVD) is the leading cause of death globally. Cardiovascular disease (CVD) is a class of diseases that involve the heart or blood vessels and includes coronary artery disease, stroke, heart failure, cardiomyopathy, congenital heart disease and venous thrombosis. There are many risk factors for CVD including age, high blood pressure, smoking, poor diet, excessive alcohol consumption and/or a lack of exercise.

Neurodegenerative diseases or conditions include, for example, spinal cord injury, Alzheimer's disease, Parkinson disease, stroke, acute traumatic injury, amyotrophic lateral sclerosis (ALS). Treatment of these diseases or conditions requires neuro-regeneration.

Spinal cord injury is defined as damage to the spinal cord that causes a change in its function. This change may be temporary or permanent. The symptoms of the injury will depend on what level on the spinal cord the injury has occurred. Neuro-regeneration is one possible avenue for the treatment of spinal cord injury.

There is a spectrum of diseases associated with compromised epithelial and/or endothelial barrier function. Compromised barrier function is damage in either the endothelial or epithelial cell layers which opens tight junctions, thus increasing the distance between cells, leading to an increase in permeability, for instance, an increase in permeability of blood vessels or the gut. The endothelial barrier acts as a selective barrier between the blood vessels and the surrounding tissue. Diseases with compromised endothelial barrier function include, for example, cerebral malaria, acute respiratory distress syndrome, Ebola, Marburg virus infection, swine fever or African swine fever. Diseases with compromised epithelial barrier function include, for example, asthma, ulcerative colitis, Crohn's disease, chronic diarrheal disease, and Cholera.

Peripheral neuropathy may be chronic or acute and refers to nerve damage. It may be caused by a disease or by trauma. Pain and parathesia are common among sufferers.

Angiotensin II (Ang II) is a peptide hormone that causes vasoconstriction and an increase in blood pressure and is a regulator of aldosterone secretion. It plays an important role in the renin-angiotensin system. In the body, angiotensin I is converted to Ang II through removal of two C-terminal residues by angiotensin converting enzyme (ACE). Ang II acts through at least two types of receptors termed angiotensin II receptor type 1 (AT1) and angiotensin II receptor type 2 (AT2). Ang II plays a role in the nervous system and cardiovascular functions with fatal detrimental effects under pathophysiological conditions when stimulating AT1.

AT2 is a G protein-coupled receptor encoded by the AGTR2 gene. Various peptide and non-peptide agonists of AT2 have been disclosed in the literature.

Many groups have tried to model non-peptidic agonists against Ang II with favourable preference to AT2, but have been largely unsuccessful.

U.S. Pat. No. 9,707,268 teaches a cyclic octapeptide having an internal thioether linkage and based on Ang II peptide, for use as a medicament. Other groups have disclosed tri-cyclic compounds for use as AT2 receptor agonists (U.S. Pat. Nos. 8,124,638, 8,080,571, 8,067,418, 7,652,054). U.S. Pat. No. 6,444,646 teaches the use of an Ang I-like peptide for accelerating would healing.

WO2016139475 discloses to the use of compounds that are Ang II receptor agonists, e.g. AT2 agonists, for treating pulmonary fibrosis and WO1999/063930 discloses compounds that bind to angiotensin receptors.

Ranjit Singh Padda et al., (J Diabetes Metab. 2015) discusses Ang-(1-7) to treat hypertension and nephropathy in a diabetes patient.

It is an object of the invention to overcome at least one of the above-referenced problems and to provide an improved treatment for cardiovascular diseases, neurodegenerative disease, diseases with compromised barrier function or cancer treatment.

### SUMMARY OF THE INVENTION

One aspect of the current invention provides a peptide comprising (or consisting of) of an amino acid sequence of SEQUENCE ID NO. 1, in which the sequence has 1, 2, or 3 amino acid changes (herein referred to the “peptide of the invention”).

In a preferred embodiment, the sequence has 1 to 3 amino acid changes.

The term “peptide of the invention” should also be taken to include a peptide comprising (or consisting of) SEQUENCE ID NO. 1.

The peptide may be man-made or recombinant.

In an embodiment, the peptide of the invention comprises a maximum of 6 amino acids.

In an embodiment, the sequence of the peptide of the invention comprises an N-terminal amino acid extension. This extension may be 1 to 3 amino acids in length, preferably 2 amino acids in length.

In an embodiment, the sequence of the peptide of the invention comprises a C-terminal amino acid extension. This extension may be 1 to 3 amino acids in length, preferably 2 amino acids in length.

In a preferred embodiment, the peptide of the invention comprises (or consists of) an amino acid sequence of any one of SEQUENCE ID NO. 2 to 8.

A further aspect of the invention provides a composition (hereinafter “composition of the invention”) comprising an effective amount of a peptide of the invention.

The composition may also comprise a peptide comprising (or consisting of) SEQUENCE ID NO. 1.

Typically, the composition of the invention comprises a therapeutically effective amount of a peptide of the invention.

The composition of the invention may be a pharmaceutical composition.

Typically, the composition of the invention further comprises at least one pharmaceutically acceptable excipient or additive or carrier.

Preferably, the composition of the invention further comprises at least one pharmaceutically acceptable active.

The peptide of the invention may be a modified peptide. The peptide may be modified at the N-terminus and/or C-terminus. The modification may be acetylation and/or amination. The peptide may have N-acetylation and/or C-amidation.

The peptide of the invention may be a conjugate.

A further aspect of the invention provides a conjugate (hereinafter “conjugate of the invention”) comprising a peptide of the invention.

Typically, the peptide of the invention is an agonist of AT2 receptor.

A further aspect of the invention provides an antagonist of the AT2 receptor. The receptor antagonist is a peptide comprising (or consisting of) SEQUENCE ID NO. 2, or a functional variant or fragment thereof. The peptide may be a functional fragment or variant of SEQUENCE ID NO. 2.

A further aspect of the invention provides a peptide of the invention for use as a medicament.

A further aspect of the invention provides a peptide comprising (or consisting of) SEQUENCE ID NO. 1 for use as a medicament.

A further aspect of the invention provides a conjugate of the invention for use as a medicament.

A further aspect of the invention provides a composition of the invention for use as a medicament.

A still further aspect provides a peptide of the invention, the composition of the invention or the conjugate of the invention, for use in a method of treating or preventing a cardiovascular disease.

Preferably, the cardiovascular disease is selected from the group comprising coronary artery disease, cerebrovascular disease, peripheral arterial disease, atherosclerosis, arteriosclerosis, cardiac hypertrophy, heartfailure, stroke, hypertension, myocardial infarction, erectile dysfunction, diabetic cardiomyopathy, chronic heart failure (including congestive heart failure, diastolic heart failure and systolic heart failure), acute heart failure, ischemia, recurrent ischemia, arrhythmias, angina (including exercise-induced angina, variant angina, stable angina, unstable angina), acute coronary syndrome, Duchene muscular dystrophy, myocarditis, dilated cardiomyopathy, Marfan, right ventricular failure, congenital heart disease, cerebral malaria, and venous thrombosis.

Preferably, the cardiovascular disease is selected from the group comprising peripheral arterial disease, stroke, Marfan, Duchene muscular dystrophy, and cerebral malaria.

A still further aspect provides a peptide of the invention, the composition of the invention or the conjugate of the invention for use in a method of treating or preventing a neurodegenerative disease or condition.

Preferably, the neurodegenerative disease or condition is selected from the group comprising spinal cord injury, Alzheimer's disease (AD), dementias, Parkinson disease (PD), PD-related disorders including vascular PD, Huntington's Disease (HD), stroke, acute traumatic injury, amyotrophic lateral sclerosis (ALS), prion disease, motor neurone diseases (MND), spinocerebellar ataxia (SCA), spinal muscular atrophy (SMA), Batten disease, diffuse Lewy body disease, traumatic brain injury, Niemann-Pick disease, Hallervorden-Spatz syndrome, neuroaxonal dystrophy and multiple system atrophy, Pick's disease, Rett syndrome, corticobasal degeneration, progressive supranuclear palsy, frontotemporal dementia.

Preferably, the neurodegenerative disease or condition is selected from the group comprising spinal cord injury, Alzheimer's disease (AD) and motor neurone diseases (MND).

A still further aspect provides a peptide of the invention, the composition of the invention or the conjugate of the invention for use in a method of treating or preventing a disease associated with compromised endothelial and/or epithelial barrier function.

Preferably, the disease associated with compromised epithelial barrier function include, but are not limited to, diseases caused by microorganisms, such as Vibrio cholera, Clostridium perfringens, Clostridium difficile, Helicobacter pylori, asthma, ulcerative colitis, Crohn's disease, chronic diarrheal disease, psoriasis, encephalitis, diabetes (diabetic retinopathy), cancer such as epithelial tumours, urinary bladder carcinoma and colon carcinomas, atopic dermatitis, inflammatory bowel disease, and intestinal disorders.

Diseases with compromised endothelial barrier function include, for example, cerebral malaria, acute respiratory distress syndrome, Ebola, Marburg virus infection, swine fever or African swine fever, cancer, chronic inflammation, diabetes mellitus, acute lung injury or disease, vascular disease, such as coronary artery disease (atherosclerosis), hypertension, hypercholesterolemia, inflammatory disease such as rheumatoid arthritis and systemic lupus erythematosus.

A still further aspect provides a peptide of the invention, the composition of the invention or the conjugate of the invention for use in a method of treating or preventing pain. In a preferred embodiment, said pain is that caused by, or associated with, peripheral neuropathy. Typically, said pain is chronic pain or chronic peripheral pain. In a preferred embodiment, the peptide is an antagonist of the AT2 receptor. Preferably, the peptide comprises (or consists of) a sequence of SEQUENCE ID NO. 2 A still further aspect provides a peptide of the invention, the composition of the invention or the conjugate of the invention for use in a method of treating or preventing cancer. Preferably, cancer is kidney cancer.

In one embodiment, the composition of the invention is administered systemically.

In one embodiment, the composition of the invention (especially a pharmaceutical composition) is formulated for oral or parenteral administration. Other methods of administration are described herein.

A further aspect of the current invention relates to a man-made treatment composition comprising the composition of the invention or the peptide of the invention.

Preferably, the composition or product is man-made.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, the term “cardiovascular disease” refers to diseases or disorders of the cardiovascular system, including disorders of the arteries, veins and capillaries. These diseases involve an inflammation and/or accumulation plaque. Cardiovascular diseases include, but are not limited to, coronary artery disease, cerebrovascular disease, peripheral arterial disease, atherosclerosis, arteriosclerosis, cardiac hypertrophy, heart failure, stroke, hypertension, myocardial infarction, erectile dysfunction, diabetic cardiomyopathy, heart failure (including congestive heart failure, diastolic heart failure and systolic heart failure), acute heart failure, ischemia, recurrent ischemia, arrhythmias, angina (including exercise-induced angina, variant angina, stable angina, unstable angina), acute coronary syndrome, Duchene muscular dystrophy, myocarditis, dilated cardiomyopathy, Marfan, right ventricular failure, congenital heart disease, cerebral malaria, and venous thrombosis. The cardiovascular disease may be one associated with or resulting from renal disease, diabetes, or vascular calcification. Additional examples of cardiovascular diseases are known in the art.

As used herein, the term “neurodegenerative disease or condition” refers to a disease or disorder which primarily affects the neurons in the brain, including progressive loss of structure or function of neurons, including death of neurons. Neurodegenerative diseases include, but are not limited to, spinal cord injury, Alzheimer's disease (AD), dementias, Parkinson disease (PD), PD-related disorders including vascular PD, Huntington's Disease (HD), stroke, acute traumatic injury, amyotrophic lateral sclerosis (ALS), prion disease, motor neurone diseases (MND), spinocerebellar ataxia (SCA), spinal muscular atrophy (SMA), Batten disease, diffuse Lewy body disease, traumatic brain injury, Niemann-Pick disease, Hallervorden-Spatz syndrome, neuroaxonal dystrophy and multiple system atrophy, Pick's disease, Rett syndrome, corticobasal degeneration, progressive supranuclear palsy, frontotemporal dementia. Additional examples of neurodegenerative diseases are known in the art. Neurodegenerative disease may also refer to “diseases requiring neuro-regeneration”. The neurodegenerative disease may also include spinal cord injury.

When used herein the term “cancer” may be selected from the group comprising but not limited to, gastrointestinal cancer, head and neck cancer, cancer of the nervous system, kidney cancer, renal cell carcinoma, retinal cancer, melanoma, stomach cancer, liver cancer, genital-urinary cancer, colorectal cancer, and bladder cancer, multiple myeloma, glioblastoma, lymphoma, fibrosarcoma; myxosarcoma; liposarcoma; chondrosarcom; osteogenic sarcoma; chordoma; angiosarcoma; endotheliosarcoma; lymphangiosarcoma; lymphangio-endotheliosarcoma; synovioma; mesothelioma; Ewing's tumour; leiomyosarcoma; rhabdomyosarcoma; colon carcinoma; pancreatic cancer; breast cancer; ER-positive breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumour; cervical cancer; uterine cancer; testicular tumour; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; retinoblastoma; and leukemias, epithelial cancer, solid tumour and haematological malignancies, metastatic cancer. Typically, treatment of the cancer entails reducing one or more of survival, proliferation and migration of, or invasion by, cancer cells.

When used herein “compromised barrier function” refers to a barrier such as the endothelial or epithelial barrier in any part of the body, in which normal barrier function is impaired.

Preferably, the disease associated with compromised epithelial barrier function include, but are not limited to, diseases caused by microorganisms, such as Vibrio cholera, Clostridium perfringens, Clostridium difficile, Helicobacter pylori, asthma, ulcerative colitis, Crohn's disease, chronic diarrheal disease, psoriasis, encephalitis, diabetes (diabetic retinopathy), cancer such as epithelial tumours, urinary bladder carcinoma and colon carcinomas, atopic dermatitis, inflammatory bowel disease, and intestinal disorders.

Diseases with compromised endothelial barrier function include, for example, cerebral malaria, acute respiratory distress syndrome, Ebola, Marburg virus infection, swine fever or African swine fever, cancer, chronic inflammation, diabetes mellitus, acute lung injury or disease, vascular disease, such as coronary artery disease (atherosclerosis), hypertension, hypercholesterolemia, inflammatory disease such as rheumatoid arthritis and systemic lupus erythematosus.

The term “pain” refers to a physical sensation in a person or patient caused by illness or injury. Pain includes neuropathic pain, nociceptive pain, psychogenic pain, visceral pain or a combination thereof.

The term “chronic pain” is pain that persists beyond normal healing time. In general, chronic pain is that which lasts longer than 12 weeks. The causes of chronic pain may include by are not limited to headaches, migraine, musculoskeletal, neurological, psychological, or disease related.

The term “peripheral neuropathy” is a condition that develops as a result of damage to the peripheral nervous system. It includes mononeuropathies, polyneuropathies and chronic neuropathies. Symptoms vary depending on whether motor, sensory, or autonomic nerves are damaged. Symptoms may include pain and voluntary movement of muscle. Peripheral neuropathy may be inherited or acquired through disease, including neurodegenerative disease, or trauma.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In this specification, the term “composition” should be understood to mean something made by the hand of man, and not including naturally occurring compositions.

“Pharmaceutical compositions”: A further aspect of the invention relates to a pharmaceutical composition comprising a peptide of the invention or a composition of peptides of the invention, admixed with one or more pharmaceutically acceptable diluents, excipients or carriers. Even though the peptides and compositions of the present invention can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients”, 2^{nd} Edition, (1994), edited by A Wade and PJ Weller. In particular, formulations for topical delivery are described in “Topical Drug Delivery Formulations” edited by David Osborne and Antonio Aman, Taylor & Francis, the complete contents of which are incorporated herein by reference. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of phydroxybenzoic acid. Antioxidants and suspending agents may be also used.

The term “mammal” should be understood to mean a higher mammal, especially a human. However, the term also includes non-mammalian animals such as fish. The human may be an infant, toddler, child, adolescent, adult, or elderly human.

The term “peptide” used herein refers to a polymer composed of amino acids, for example 1 to 6 amino acid monomers typically linked via peptide bond linkage. Peptides (including fragments and variants thereof) of and for use in the invention may be generated wholly or partly by chemical synthesis or by expression from nucleic acid. For example, the peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Ill. (1984); and M. Bodanzsky and A. Bodanzsky, The Practice of Peptide Synthesis, Springer Verlag, New York (1984)). When necessary, any of the peptides employed in the invention can be chemically modified to increase their stability. A chemically modified peptide or a peptide analog includes any functional chemical equivalent of the peptide characterized by its increased stability and/or efficacy in vivo or in vitro in respect of the practice of the invention.

The term “peptide analog” also refers to any amino acid derivative of a peptide as described herein. A “peptide analog” may be used interchangeably with the term “modified peptide”. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tyrosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Peptide structure modification includes the generation of retro-inverso peptides comprising the reversed sequence encoded by D-amino acids. Changes may be those that reduce susceptibility to proteolysis, reduce susceptibility to oxidation, alter binding affinity of the variant sequence (typically desirably increasing affinity), and/or confer or modify other physicochemical or functional properties on the associated variant/analog peptide.

The “peptide of the invention” when used herein refers to a polymer composed of amino acids, for example 6 amino acid monomers, typically linked via peptide bond linkage and having an amino acid sequence of SEQUENCE ID NO: 1. It also includes amino acid sequences that are substantially identical to SEQUENCE ID NO. 1, but altered in respect of one or more amino acid residues, for example alteration of 1, 2, or 3 residues (hereafter “variants” or “peptide variants” or “functional variants”). Preferably, such alterations involve the substitution of 3 or fewer amino acids, most preferably of 1 or 2 amino acids only. Substitution with natural and modified amino acids is envisaged. The peptide may have conservative amino acid changes, wherein the amino acid being introduced is similar structurally, chemically, or functionally to that being substituted. The change or alteration may be a deletion. The peptide may have non-conservative amino acid changes. The peptide may be a maximum of 6 amino acids in length.

The term “peptide of the invention” should be understood to also include a fragment of amino acid sequence of SEQUENCE ID NO. 1 (herein “fragment” or “functional fragment”). The “functional fragment” may be from 3 to 6 amino acids in length, preferably 4, or 5 amino acids in length. Generally, the fragment has a charge of −5 to +3. The charge of a peptide, fragment or region is determined using the method of Cameselle, J. C., Ribeiro, J. M., and Sillero, A. (1986). Derivation and use of a formula to calculate the net charge of acid-base compounds. Its application to amino acids, proteins and nucleotides. Biochem. Educ. 14, 131-136.

The term “functional” variant or “functional” fragment refers to a variant or fragment of the peptide of the invention and which is capable of acting as an agonist of AT2 in the methods as described herein and/or capable of treating or preventing cardiovascular disease or neurodegenerative diseases. The term “functional” variant or “functional” fragment also refers to a variant or fragment of the peptide of the invention and which is capable of acting as an antagonist of AT2 in the methods as described herein and/or capable of treating or preventing pain, e.g. a functional variant or fragment of SEQUENCE ID NO. 2.

In this specification, the term “sequence identity” should be understand to comprise both sequence identity and similarity, i.e. a variant (or homolog) that shares 70% sequence identity with a reference sequence is one in which any 70% of aligned residues of the variant (or homolog) are identical to, or conservative substitutions of, the corresponding residues in the reference sequence across the entire length of the sequence. Sequence identity is the amount of characters, which match exactly between two different sequences. The measurement is relational to the shorter of the two sequences.

In terms of “sequence homology”, the term should be understood to mean that a variant (or homolog) which shares a defined percent similarity or identity with a reference sequence when the percentage of aligned residues of the variant (or homolog) are either identical to, or conservative substitutions of, the corresponding residues in the reference sequence and where the variant (or homolog) shares the same function as the reference sequence.

This alignment and the percent homology or sequence identity can be determined using software programs known in the art, for example, one alignment program is BLAST, using default parameters. Details of these programs can be found at the following Internet address: http://www.ncbi.nlm.nih.gov/blast/Blast.cgi.

“C-terminal domain” as applied to a fragment means the first three amino acids at the c-terminus of the fragment.

“N-terminal domain” as applied to a fragment means the last three amino acids at the n-terminus of the fragment.

The term “recombinant” when used herein will be understood as referring to biological material containing or derived from genetic material of more than one origin. It is a manipulated form of biological material produced by recombinant technology. For example, a recombinant peptide would generally be understood to be derived from a polynucleotide molecule encoding the peptide of a first origin that is expressed or produced in a suitable host cell of a second origin. A wide range of expression systems that can produce recombinant peptides are known, for example, in cell cultures under conditions suitable for the expression of the particular peptide. Recombinant peptides can alternatively be expressed in transgenic plants and animals.

In the specification, the terms “comprise, comprises, comprised and comprising” or any variation thereof and the terms “include, includes, included and including” or any variation thereof are considered to be totally interchangeable and they should all be afforded the widest possible interpretation and vice versa.

### BRIEF DESCRIPTION OF THE FIGURES

The invention will be more clearly understood from the following description of an embodiment thereof, given by way of example only, with reference to the accompanying drawings, in which:

FIG. 1 illustrates increased intracellular cAMP levels after Ang (1-6) stimulation in AT2-transfected HEK293 cells. (A) cAMP concentration in AT2-transfected HEK293 after stimulation for 15 min with a range of concentrations (10⁻¹⁴ to 10⁻⁵ M) of the non-peptitic AT2 agonist C21 (in grey) as a positive control, Ang II (in black) or solvent (white bars); (B) cAMP concentration in pcDNA-transfected or (C) AT2-transfected HEK293 following stimulation for 15 min with a range of concentrations (10⁻¹⁶ to 10⁻⁶ M) of Ang-(1-6); (D) cAMP concentration in AT2-transfected HEK293 following stimulated for 15 min with blockers (D-Ala-Ang-(1-7) (A779), D-Pro⁷-Ang-(1-7) (D-Pro) and PD123319 (all 10⁻⁶ M)), followed by 15 min stimulation with Ang-(1-6) (EA) (10⁻¹¹M) or C21 (10⁻⁷M); (E) cAMP concentration in Mas transfected or (f) MrgD-transfected HEK293 following stimulation for 15 min with a range of concentrations (10⁻¹⁴ to 10⁻⁷ M) of Ang-(1-6). Results are expressed as mean±SEM. Data was reported as a fold change or percentage of the untreated control mean. *P<0.05, **P<0.01, ***P<0.001, significantly different from control (media); ^{###}P<0.001, or ^{#}P<0.05, significantly different to C21 alone; ^{$$$}P<0.001 significantly different from Ang-(1-6) alone; ANOVA with Bonferroni post-hoc test.

FIG. 2 illustrates signalling mechanisms of Ang-(1-6) showing adenylyl cyclase dependency but no Galpha I involvment, and shows effects of Ang-(1-6) in HUVEC and Mesangial cells. (A) AT2-transfected HEK293 cells were stimulated for 6 h with the Galpha i inhibitor PTX (50 ng/ml) followed by stimulation with Ang-(1-6) (10⁻¹¹ M) for a further 15 mins; (B) AT2-transfected HEK293 cells were stimulated for 15 min with the adenylyl cyclase inhibitor SQ-22536 (2×10⁻⁶ M), followed by 15 min stimulation with Ang-(1-6) (10⁻¹¹ M); (C) HUVEC were stimulated for 15 min with a range of concentrations (10⁻¹³ to 10⁻⁶ M) of C21 or (D) Ang-(1-6) (10⁻¹⁴ to 10⁻⁶ M) before analysis of cAMP concentration; (E) AT2 KO primary mesangial cells or (F) WT mesangial cells were stimulated for 15 min with a range of concentrations (10⁻¹³ to 10⁻⁷ M) of Ang-(1-6), or (G) cells were pre-stimulated for 15 min with blockers (A779, D-Pro⁷-Ang-(1-7) (D-Pro) and PD123319 (all 10⁻⁶ M)), followed by 15 min stimulation with Ang-(1-6) 10⁻¹⁰ M. P<0.05, **P<0.01,***P<0.001, significantly different from control (media); ^{###}P<0.001, or ^{##}P<0.01, significantly different to Ang-(1-6); ANOVA with Bonferroni post-hoc test.

FIG. 3 (A) to (C) illustrates luciferase production in HEK293 cells transiently co-transfected with pcDNA3.1, AT2, orAT1, and pNFAT-Luc, pCREB-Luc or pElk1-Luc after stimulation with PBS, C21, Ang II or Ang-(1-6). Results are expressed as mean±SEM. Data was reported as a fold change or percentage of the untreated control mean. **P<0.01 ***P<0.001 significantly different from media-stimulated receptor transfected control.

FIG. 4 illustrates rapid Ang-(1-6) peptide degradation, and in silico modelling showing perfect fit into the AT2 receptor. (A) Ang-(1-6) degradation in mouse kidney membranes after 15 minutes compared to Ang-(1-7) and Ang II, (B) Ang-(1-6), Ang II and the PDB 5UNG ligand were docked into the binding site of the AT2 receptor. Gliding scores of the ligands into the receptor are listed in the table.

FIG. 5 illustrates Ang-(1-6) protective effect on epithelial and endothelial cell function. (A) HRMECs were treated with Ang-(1-6) or PBS (vehicle) for 15 mins, experiment was repeated with new media containing VEGF (50 ng/ml) or PBS (vehicle). xCelligence data was exported to Prism for analysis (diagram), results are expressed as mean±SEM. *P<0.05, ANOVA with Bonferroni post-hoc test; (B) Mesenteric arteries were contracted with NA (1 μmol/L). Artery relaxation was assessed by adding cumulative concentrations of Ang-(1-6), C21, at 2 min intervals (final bath concentrations 0.1 nmol/L to 10 μmol/L) solvent (PBS) acted as control, C. Intestinal preparations of wild-type mice were mounted in Ussing chambers. Pertussis toxin (toxin) was used to compromise barrier function, and Ang-(1-6) and peptide modifications (10⁻¹¹ M) were added to test for their effect on restoring barrier function (FIG. 5C).

FIG. 6 illustrates intracellular cAMP levels after Ang-(1-7) stimulation in AT2-transfected HEK293 cells. cAMP concentration in AT2-transfected HEK293 after stimulation for 15 min with C21(10⁻⁷ M), or with a range of concentrations (10⁻¹⁴ to 10⁻⁶ M) of Ang (1-7). ***P<0.001, significantly different from control (media).

FIG. 7 illustrates intracellular cAMP levels after Ang-(1-4) stimulation in AT2-transfected HEK293 cells. cAMP concentration in AT2-transfected HEK293 after stimulation for 15 min with C21(10⁻⁷ M), or with a range of concentrations (10⁻¹⁴ to 10⁻⁶ M) of Ang-(1-4). **P<0.01, significantly different from control (media).

FIG. 8 illustrates an increase in intracellular cAMP levels after Ang-(1-6) stimulation, no effect of Ala¹-Ang-(1-6) stimulation, but an antagonizing effect of increasing concentration of Ala¹-Ang-(1-6). (A) AT2-transfected HEK293 cells were stimulated with Ang-(1-6) (10⁻¹¹ or 10⁻¹⁰ M) and the effect of Ang-(1-6) (10⁻¹⁰ M) blocked by increasing concentrations of Ala-Ang-(1-6) (10⁻¹³ to 10⁻⁸ M); B. AT2-transfected HEK293 cells were stimulated with increasing concentrations of Ala¹-Ang-(1-6) (10⁻¹¹ to 10⁻⁷ M), whereby Ang-(1-6) (10⁻¹¹ or 10⁻¹⁰ M) was used as a positive control. C. Stimulatory effect of Ang-(1-6) (10⁻¹⁰ M) was blocked by increasing concentrations of Ala-Ang-(1-6) (10⁻¹³ to 10⁻⁸ M). *P<0.05, **P<0.01, compared to solvent control (media); ^{##}P<0.01, ^{###}P<0.001, compared to Ang-(1-6); ANOVA with Bonferroni post-hoc test.

FIG. 9 illustrates intracellular cAMP levels after Ala¹-Ang-(1-6) stimulation, C21 stimulation or stimulation with a range of concentrations of Ala¹-Ser⁴-Ang-(1-6) (10⁻¹⁴ to 10⁻⁷ M). *P<0.05, significantly different from control (media).

FIG. 10 illustrates intracellular cAMP levels after Ang-(1-6) stimulation, or stimulation with a range of concentrations of Ser⁴-Ang-(1-6) (10⁻¹⁵ to 10⁻⁷ M). *P<0.05, significantly different from control (media).

FIG. 11 illustrates the number of colonies formed under treatment of Ang-(1-6) or its peptide modifications. The results of a colony formation assay using Ang (1-6) (10⁻¹¹M), and Ang (2-6) (10⁻¹¹M), and Ser⁴-Ang (2-6) (10⁻¹⁴ to 10⁻⁹ M). *P<0.05, **P<0.01, compared to solvent control (PBS).

### DETAILED DESCRIPTION OF THE DRAWINGS

The invention will now be described with reference to specific examples. These are merely exemplary and for illustrative purposes only: they are not intended to be limiting in any way to the scope of the monopoly claimed or to the invention described. These examples constitute the best mode currently contemplated for practicing the invention.

In the broadest sense, the first aspect of the invention provides a peptide comprising (or consisting of) an amino acid sequence of SEQUENCE ID NO. 1 (Also referred to as Ang (1-6)).

It is preferred that the peptide comprises (or consists of) an amino acid sequence of SEQUENCE ID NO, 1. In which the sequence has 1, 2, 3, or 4 amino acid changes. SEQUENCE ID NO. 1 has the following sequence:

DRVYIH.

Preferably, the peptide has a maximum length of 6 amino acids. Typically, the peptide has a length of 6 amino acids.

The peptide may consist essentially of SEQUENCE ID NO. 1.

The inventors have discovered that a peptide, Ang-(1-6), a hexamer, assumed to be an inactive Ang catabolite, having an amino acid sequence of SEQUENCE ID NO. 1, is an agonist of the angiotensin type II receptor, AT2. This activation increases the concentration of intracellular cAMP. Ang-(1-6) is an endogenous agonist for the AT2 receptor and is a natural metabolite of either Ang II or Ang-(1-7), whereby Ang II cannot activate G-proteins via the receptor and Ang-(1-7) not stimulate the receptor at all.

The current invention also includes a pharmaceutical composition comprising the peptide of the invention.

In an embodiment of the invention, the peptide is a modified peptide. The modification may be a modification as described and defined here. In an embodiment of the invention, the peptide is a conjugate. In other words, in one embodiment, the invention provides a conjugate comprising a peptide of the invention.

The amino acid sequence of the peptide of the invention may have an N-terminal amino acid extension. The amino acid sequence of the peptide of the invention may have a C-terminal amino acid extension

The invention further provides the peptide of the invention or the composition of the invention for use as a medicament.

In an embodiment, the current invention also provides the peptide of the invention or the composition of the invention for use in a method of treating or preventing cardiovascular disease. The cardiovascular disease includes, but is not limited to cardiovascular disease, is selected from the group comprising coronary artery disease, cerebrovascular disease, peripheral arterial disease, atherosclerosis, arteriosclerosis, cardiac hypertrophy, heart failure, stroke, hypertension, myocardial infarction, erectile dysfunction, diabetic cardiomyopathy, chronic heart failure (including congestive heart failure, diastolic heart failure and systolic heart failure), acute heart failure, ischemia, recurrent ischemia, arrhythmias, angina (including exercise-induced angina, variant angina, stable angina, unstable angina), acute coronary syndrome, Duchene muscular dystrophy, myocarditis, dilated cardiomyopathy, Marfan, right ventricular failure, congenital heart disease, cerebral malaria, and venous thrombosis.

Preferably, the cardiovascular disease is selected from the group comprising peripheral arterial disease, stroke, Duchene muscular dystrophy and cerebral malaria.

The current invention also provides the peptide of the invention or the composition of the invention for use in a method of treating or preventing a neurodegenerative disease or condition. The neurodegenerative disease or condition includes but is not limited to spinal cord injury, Alzheimer's disease (AD), dementias, Parkinson disease (PD), PD-related disorders including vascular PD, Huntington's Disease (HD), stroke, acute traumatic injury, amyotrophic lateral sclerosis (ALS), prion disease, motor neurone diseases (MND), spinocerebellar ataxia (SCA), spinal muscular atrophy (SMA), Batten disease, diffuse Lewy body disease, traumatic brain injury, Niemann-Pick disease, Hallervorden-Spatz syndrome, neuroaxonal dystrophy and multiple system atrophy, Pick's disease, Rett syndrome, corticobasal degeneration, progressive supranuclear palsy, frontotemporal dementia.

Preferably, the neurodegenerative disease or condition is selected from the group comprising spinal cord injury, Alzheimer's disease (AD) and motor neurone diseases (MND).

The current invention also provides the peptide of the invention or the composition of the invention for use in a method of treating or preventing a disease associated with compromised epithelial and/or endothelial barrier function.

The current invention also provides the peptide of the invention or the composition of the invention for use in a method of treating or preventing cancer. The peptide used may be one consisting of a sequence of SEQUENCE ID NO. 1. The cancer may be kidney cancer. The cancer may be a kidney-cancer related cancer. The cancer may be one or more cancer as defined herein. The kidney cancer may be small cell renal cell carcinoma (scRCC).

The method or peptide for use in said method may be used either alone, or in conjunction with other treatment methods known to a person skilled in the art. For example, such methods may include, but are not limited to, chemotherapy, radiation therapy, or surgery.

The current invention also provides the peptide of the invention or the composition of the invention for use in treating or preventing chronic peripheral neuropathic pain. In such a method, it is preferred that the peptide used is one comprising or consisting of a sequence of SEQUENCE ID NO. 2.

In one embodiment, the peptide of the invention or the composition of the invention is to be administered with a substance or agent that allows oral delivery of the peptide, for example a cyclodextran or its derivative. In this regard, the cyclodextran is used as a complexing agent and a peptide-cyclodextran complex is formed. It is to be understood that any cyclodextran or cyclodextran derivative may be used and such are known in the art. It will be appreciated that other, or similar, agents may be used provided that they are suitable to allow oral delivery of the peptide and such agents are known in the art. The peptide or composition of the invention may be delivered by any delivery means as described herein.

The peptide of the invention may be expressed in a plant or similar by a method known in the art to a person skilled in the art, e.g. Chloroplast expression to enable encapsulation in plant cells. The plant or similar may be administered orally, i.e. ingested, by a patient in need thereof.

A method for treating or preventing cardiovascular disease is also provided. The method comprises a step of administering a therapeutically effective amount of a peptide of the invention or the composition of the invention to a patient in need thereof.

A method for treating or preventing a neurodegenerative condition or disease is also provided. The method comprises a step of administering a therapeutically effective amount of a peptide of the invention or the composition of the invention to a patient in need thereof.

A method for treating or preventing a foot ulcer, especially a diabetic foot ulcer, is also provided. The method comprises a step of administering a therapeutically effective amount of a peptide of the invention or the composition of the invention to a patient in need thereof. In one embodiment, the peptide of the invention includes norleucine at position 3.

A method for treating or preventing chronic peripheral neuropathic pain is also provided. The method comprises a step of administering a therapeutically effective amount of a peptide of the invention or the composition of the invention to a patient in need thereof. The peripheral neuropathic pain may be that associate with or caused by Charcot-Marie-Tooth neuropathy.

A method for treating or preventing a disease associated with compromised epithelial and/or endothelial barrier function is provided. The method comprises a step of administering a therapeutically effective amount of a peptide of the invention or the composition of the invention to a patient in need thereof.

A method for treating or preventing cancer is provided. The method comprises a step of administering a therapeutically effective amount of a peptide of the invention or the composition of the invention to a patient in need thereof. Typically, the cancer is kidney or kidney related cancer. The cancer may be any cancer defined herein.

As defined above, the peptide of the invention includes a peptide having a sequence of SEQUENCE ID NO. 1, in which the sequence comprises 1 to 4 amino acid changes. The sequence may comprise up to 6 amino acids in length. The sequence is altered in respect of one or more amino acid residues, for example alteration of 1, 2 or 3 residues. Preferably, such alterations involve the insertion, addition, deletion and/or substitution of 3 or fewer amino acids, most preferably of 1 or 2 amino acids only. Insertion, addition and substitution with natural and modified or non-naturally occurring, amino acids is envisaged. The peptide may have conservative amino acid changes, wherein the amino acid being introduced is similar structurally, chemically, or functionally to that being substituted. The peptide may have non-conservative amino acid changes.

In one embodiment, the peptide has 1 to 3 amino acid changes compared to SEQUENCE ID NO: 1. In one embodiment, the peptide has 1 to 2 amino acid changes compared to SEQUENCE ID NO: 1. In one embodiment, the amino acid change is a conservative amino acid change. In one embodiment, the amino acid change is an amino acid substitution. In one embodiment, the amino acid substitution is a conservative substitution. In one embodiment, the amino acid change is an amino acid addition. In one embodiment, the amino acid change is an amino acid deletion.

Preferably, the amino acid at position 1 is changed compared to SEQUENCE ID NO: 1. In an embodiment, the amino acid at position 2 is changed compared to SEQUENCE ID NO: 1. In an embodiment, the amino acid at position 3 is changed compared to SEQUENCE ID NO: 1. In an embodiment, the amino acid at position 4 is changed compared to SEQUENCE ID NO: 1. In an embodiment, the amino acid at position 5 is changed compared to SEQUENCE ID NO: 1. In an embodiment, the amino acid at position 6 is changed compared to SEQUENCE ID NO: 1. In an embodiment, the amino acid at position 1, 2, 3, 4, 5, or 6 or a combination thereof is changed compared to SEQUENCE ID NO: 1.

Preferably, the amino acid change is a substitution. The substitution may be any amino acid change.

In one embodiment, the amino acid at position 1 of the peptide sequence is altered. Preferably, this alteration is a substitution. Typically, this substitution replaces aspartic acid (D) with alanine (A). However, it will be appreciated that the amino acid at position 1 may be substituted with any known amino acid.

For example, the amino acid may be alanine, arginine, asparagine, aspartic acid, cysteine, glutamine, glutamic acid, glycine, histidine, isoleucine, leucine, norleucine, lysine, methionine, phenylalanine, proline, serine, threonine, tryptophan, tyrosine, or valine.

In one embodiment, the amino acid at position 3 is changed to norleucine. These peptides are applicable in the treatment of diabetic ulcers, especially diabetic foot ulcers.

In one embodiment, the amino acid at position 4 of the peptide sequence is altered. Preferably, this alteration is a substitution. Typically, this substitution replaces Tyrosine (Y) with Serine (Ser). However, it will be appreciated that the amino acid at position 4 may be a substitution with any known amino acid.

For example, the amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In one embodiment, the amino acid at position 1 and the amino acid at position 4 are altered. Preferably, this alteration is a substitution. The substitution may be any amino acid change. Typically, this substitution replaces aspartic acid (D) with alanine (A) and Tyrosine (Y) with Serine (Ser). However, it will be appreciated that the amino acid at position 4 may be substituted with any known amino acid. The substitution may be the same or different.

For example, the amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The peptide of the invention includes the following peptides:

(SEQUENCE ID NO. 2)
ARVYIH
(SEQUENCE ID NO. 3)
DRVSIH
(SEQUENCE ID NO. 4)
ARVSIH

As defined above, the term “peptide of the invention”, also includes a fragment or functional fragment. The fragment may be from 3 to 6 amino acids in length, preferably 4, or 5 amino acids in length.

The peptide of the invention includes the following fragment peptides: —

(SEQUENCE ID NO. 5)
RVYIH.
(SEQUENCE ID NO. 6)
RVSIH

The fragment may be a fragment of any one of the above variants. The fragment may be amino acid 2 to 6 of any one of the above variants.

In one embodiment, the amino acid at position 2 of the peptide sequence is altered. Preferably, this alteration is a substitution. Typically, this substitution replaces Arginine (R) with Lysine (K). However, it will be appreciated that the amino acid at position 2 may be substituted with any known amino acid.

For example, the amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In one embodiment, the amino acid at position 3 and the amino acid at position 5 are altered. Preferably, this alteration is a substitution. The substitution may be any amino acid change. Typically, this substitution replaces Valine (V) with Isoleucine (I) and Valine (V) with Isoleucine (I). However, it will be appreciated that the amino acid at position 3 and/or 5 may be substituted with any known amino acid. The substitution may be the same or different.

For example, the amino acid may be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The peptide of the invention includes the following peptides:

(SEQUENCE ID NO. 7)
DKVYIH
(SEQUENCE ID NO. 8)
DRIYVH

The inventors have discovered that some variants of SEQUENCE ID NO. 1 transform the agonist to an antagonist of AT2. Preferably, the variant has an amino acid change at position 1 SEQUENCE ID NO. 1, preferably this substitution replaces aspartic acid (D) with alanine (A). The variant may have an amino acid change at position 6 of SEQUENCE ID NO. 1. Blocking AT2 and thus cAMP production impacts on neuron signaling and has a beneficial effect in terms of stopping or alleviating pain in patients.

In an embodiment of the invention, the AT2 antagonist comprises a peptide comprising (or consisting of) a sequence of SEQUENCE ID NO. 2. It may be a functional variant or functional fragment of SEQUENCE ID NO. 2.

The antagonist is particularly suited for use in a method for treating or preventing pain. It will be understood that this may be any type of pain suffered by a person or patient. Preferably, said pain is chronic pain, typically caused by peripheral neuropathy.

The peptide of the invention or the composition of the invention may be incorporated into a medical device for administration. Such a device can include an implantable medical device. Such a device can include but is not limited to a stent, e.g. a vascular stent, an implantable device suitable for treatment of neurodegenerative disease including intracerebroventricular implantation, an implantable device suitable for placement under the skin of a patient.

It will be appreciated that the composition may comprise a plurality of peptides of the invention

In one aspect, the method of the invention involves treating symptoms and/or underlying conditions of cardiovascular disease and/or neurodegenerative disease.

Administration

The peptide or composition of the invention may be presented, prepared and/or administered in a variety of suitable forms. Such forms include, for example, but are not limited to, liquid, semi-solid and solid dosage forms, such as liquid solutions (e.g., injectable and infusible solutions), dispersions or suspensions, emulsions, microemulsions, tablets, pills, microneedles, powders, liposomes, dendrimers and other nanoparticles, microparticles, and suppositories. It will be appreciated that the form may depend on the intended mode of administration, the nature of the composition or combination, and therapeutic application or other intended use. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax.

In some embodiments of the current invention, the peptide or composition may be delivered via any one of liposomes, mixed liposomes, oleosomes, niosomes, ethosomes, millicapsules, capsules, macrocapsules, nanocapsules, nanostructured lipid carriers, sponges, cyclodextrins, vesicles, micelles, mixed micelles of surfactants, surfactant-phospholipid mixed micelles, millispheres, spheres, lipospheres, particles, nanospheres, nanoparticles, milliparticles, solid nanopartciles as well as microemulsions including water-in-oil microemulsions with an internal structure of reverse micelle and nanoemulsions microspheres, microparticles.

These delivery systems may be adapted to achieve a greater penetration of the compound and/or peptides of the invention. This may improve pharmacokinetic and pharmacodynamics properties. The delivery system may be a sustained release system wherein the compound or peptide of the invention is gradually released during a period of time and preferably with a constant release rate over a period of time. The delivery systems are prepared by methods known in the art. The amount of peptide contained in the sustained release system will depend on where the composition is to be delivered and the duration of the release as well as the type of the condition, disease and/or disorder to be treated or cared for.

The peptide or composition of the invention may be administered by oral administration. The compound (and other ingredients, if desired) or peptide may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. The compound may be coated, or co-administer the compound with, a material to prevent its inactivation. In another aspect, peptides or compositions of the invention orally administered, for example, with an inert diluent or an assimilable edible carrier. For oral therapeutic administration, the compounds may be incorporated with excipients and used in the form of ingestible tablets, buccal tablets, troches, capsules, elixirs, suspensions, syrups, wafers, and the like. To administer a peptide or compound of the invention by other than parenteral administration, it may be necessary to coat the compound with, or co-administer the compound with a material to prevent its inactivation.

In an embodiment, the peptide or composition of the invention may be administered by parenteral administration (e.g., intravenous, subcutaneous, intraperitoneal, and/or intramuscular administration). For example, it may be administered by intravenous infusion or injection or by intramuscular or subcutaneous injection.

In a particularly preferred embodiment, the methods and uses of the invention involve administration of a peptide or composition of the invention in combination with one or more other active agents, for example, existing growth promoting drugs or pharmacological enhancers available on the market. In such cases, the peptide or composition of the invention may be administered consecutively, simultaneously or sequentially with the one or more other active agents as e.g. growth hormones.

In a particularly preferred embodiment, the methods and uses of the invention involve administration of a peptide or composition of the invention in combination with one or more other active agents. In such cases, the compounds of the invention may be administered consecutively, simultaneously or sequentially with the one or more other active agents. The active agents are as described herein.

The composition of the invention may be for human or animal usage in human and veterinary medicine.

Compositions may be formulated in unit dosage form, i.e., in the form of discrete portions containing a unit dose, or a multiple or sub-unit of a unit dose.

A peptide of the invention can be formulated into a pharmaceutical composition as neutralized physiologically acceptable salt forms. Suitable salts include the acid addition salts (i.e., formed with the free amino groups of the peptide molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Excipients

In an embodiment of the invention the composition of the invention may further comprise at least one pharmaceutically acceptable excipient. Excipient (diluents, carriers, and the like) may be used interchangeably with functional ingredient or additive. Pharmaceutically acceptable excipient are well known in the art and any known excipient, may be used provided that it is suitable without undue toxicity, incompatibility and/or allergic reaction. A pharmaceutically acceptable excipient is one appropriate for one or more intended routes of administration to provide compositions that are pharmaceutically acceptable in the context of preparing a pharmaceutically acceptable composition comprising one or more peptides of the invention.

Preferably, any excipient included is present in trace amounts. The amount of excipient included will depend on numerous factors, including the type of excipient used, the nature of the excipient, the component(s) of the composition, the amount of active or peptide in the composition and/or the intended use of the composition. The nature and amount of any excipient should not unacceptably alter the benefits of the peptides of this invention.

In an embodiment of the invention the excipient may be a suitable diluent, carrier, binder, lubricant, suspending agent, coating agent, preservative, stabilisers, dyes, vehicle, solubilising agent, base, emollient, emulsifying agent, and/or surfactants.

A peptide of the invention may be, for example, admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and optionally further tabletted or encapsulated for conventional administration. Alternatively a peptide of the invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier or diluent may include time delay material, such as glyceryl monostearate or glyceryl distearate alone or with a wax, or other functionally similar materials.

Pharmaceutically acceptable carriers generally also include any and all suitable solvents, dispersion media, coatings, antibacterial and antifungal agents, isotonic and absorption delaying agents, and the like that are physiologically compatible with an insulin analog. Pharmaceutically acceptable substances, which desirably can enhance the shelf life or effectiveness the peptide or composition are provided.

The carrier may be any suitable carried known in the art or disclosed in US2014120131 or US2004132667. In some embodiments, the carrier may include, but is not limited to, a liquid, such as water, oils or surfactants, including those of petroleum, animal, plant or synthetic origin, polymer, oil, such as peanut oil, mineral oil, castor oil, soybean oil, alcohol, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, or digitonin. It will be understood that the carrier will be dermatologically acceptable. Preferred carriers contain an emulsion such as oil-in-water, water-in-oil, water-in-oil-in-water and oil-in-water-in-silicone emulsions. Emulsions may further contain an emulsifier and/or an anti-foaming agent.

Peptides or compositions of the invention can be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid, and combinations of any thereof, so as to provide such a composition. Methods for the preparation of such compositions are known.

Additional Ingredients

The peptide or composition of the invention may be administered individually or in combination with other pharmacologically active agents or additional ingredients. It will be understood that such combination therapy encompasses different therapeutic regimens, including, without limitation, administration of multiple agents together in a single dosage form or in distinct, individual dosage forms. If the agents are present in different dosage forms, administration may be simultaneous or near-simultaneous or may follow any predetermined regimen that encompasses administration of the different agents.

Such additional ingredients may be those of benefit to include in a composition, or of benefit depending on the intended use of the composition. The additional ingredient may be active or functional or both. If the agents are present in different dosage forms, administration may be simultaneous or near-simultaneous or may follow any predetermined regimen that encompasses administration of the different agents.

The additional ingredient may be one suitable for treatment of a cardiovascular disease or a symptom thereof. The additional ingredient may be one suitable for treatment of a neurodegenerative disease or a symptom thereof. This may be called combination therapy. Therapeutic agents suitable for treating cardiovascular related diseases or conditions include anti-anginals, heart failure agents, antithrombotic agents, antiarrhythmic agents, antihypertensive agents, antihyperglycaemic, and lipid lowering agent. Therapeutic agents suitable for neurodegenerative diseases or conditions include but are not limited to anti-depressants, Anti-spastic agents and anxiolytic agents.

The additional ingredient may be one suitable for treatment of a disease with compromised barrier function. Therapeutic agents suitable for diseases with compromised barrier function or conditions include but are not limited to antidiarrhoeal agents, and agents against human or animal haemorrhagic viruses. It will be appreciated that such agents are known in the art and all are encompassed herein. The additional ingredient may be one suitable for treatment of pain and may be any suitable ingredient known in the art.

It is to be understood that additional ingredients listed may provide more than one benefit. The classification given herein is for clarity and convenience only and not intended to limit the additional ingredient to that particular application or category listed. The additional ingredient may provide a benefit to the treatment of cardiovascular disease. The additional ingredient may provide a benefit to the treatment of a disease requiring neuro-regeneration.

It will be appreciated that a plurality of additional ingredients may be added. The amount of additional ingredient included will depend on numerous factors, including the type of additional ingredient used, the nature of the additional ingredient, the component(s) of the composition, the amount of active or peptide in the composition and/or the intended use of the composition. The nature and amount of any additional ingredient should not unacceptably alter the benefits of the peptides of this invention.

In some embodiments of the invention, the composition further comprises one or more additional active agents, in addition to the peptide of the invention (also known as the active of the composition). In addition, or alternatively, the composition may be administered with one or more other additional active agents. Typical said additional active agent is present in trace amounts only. In some embodiments, there may be no additional ingredient present in the composition. The amount of additional ingredient included will depend on numerous factors, including the type of additional ingredient used, the nature of the additional ingredient, the component(s) of the composition, the amount of ingredient or peptide in the composition and/or the intended use of the composition.

The additional ingredient may be an active agent It is to be understood that an ingredient that is considered to be an “active” ingredient in one product may be a “functional” or “excipient” ingredient in another and vice versa. It will also be appreciated that some ingredients play a dual role as both an active ingredient and as a functional or excipient ingredient.

Compositions of the invention also include compositions comprising any suitable combination of a peptide of the invention and a suitable salt therefor. Any suitable salt, such as an alkaline earth metal salt in any suitable form (e.g., a buffer salt), can be used in the stabilization of the peptide of the invention (preferably the amount of salt is such that oxidation and/or precipitation of the peptide is avoided). Suitable salts typically include sodium chloride, sodium succinate, sodium sulfate, potassium chloride, magnesium chloride, magnesium sulfate, and calcium chloride. Compositions comprising a base and one or more peptides of the invention also are provided.

Modified Peptides

In one embodiment, the peptide of the invention (including peptide variants and peptide fragments) may be a modified peptide. The term “modified peptide” is used interchangeably with the term derivative of the peptide or the term peptide analog. In one embodiment, the term “modified peptide” means a peptide that is modified to exhibit one or more of the following properties compared with the unmodified peptide: increase the plasma half-life; increase the lipophilicity of the peptide; increase the renal clearance of the modified peptide; increase the activity of the modified peptide, and increase the resistance of the modified peptide to proteolytic degradation (i.e. by mammalian and especially human gastrointestinal proteases). Various methods of modifying a peptide of the invention to exhibit these properties are disclosed herein, including conjugating the peptide with a binding partner (for example an albumin binding small molecule, large polymer, long life plasma protein, or antibody or antibody-fragment), cyclisation, addition of N- or C-terminal, or side chain, protecting groups, replacing one or more L-amino acids with D-isomers, amino acid modification, increased plasma protein binding, increased albumin binding. The modified peptide includes but is not limited to a peptide which has been substituted with one or more groups as defined herein, or conjugated with a binding partner, or cyclized. Generally, the peptide is modified to increase its half-life in vivo in an animal or human. Various methods of modification are provided below.

In one embodiment, the modification may be any modification that provides the peptides and or the composition of the invention with an increased ability to penetrate a cell. In one embodiment, the modification may be any modification that increases the half-life of the composition or peptides of the invention. In one embodiment, the modification may be any modification that increases activity of the composition or peptides of the invention. In one embodiment, the modification may be any modification that increases selectivity of the composition or peptides of the invention.

In one embodiment, the group is a protecting group. The protecting group may be an N-terminal protecting group, a C-terminal protecting group or a side-chain protecting group. The peptide may have one or more of these protecting groups.

The person skilled in the art is aware of suitable techniques to react amino acids with these protecting groups. These groups can be added by preparation methods known in the art, for example the methods as outlined in paragraphs [0104] to [0107] of US2014120141. The groups may remain on the peptide or may be removed. The protecting group may be added during synthesis.

In an embodiment of the invention the peptides may be substituted with a group selected from one or more straight chain or branched chain, long or short chain, saturated, or unsaturated, substituted with a hydroxyl, amino, amino acyl, sulfate or sulphide group or unsubstituted having from 1 to 29 carbon atoms. N-acyl derivatives include acyl groups derived from acetic acid, capric acid, lauric acid, myristic acid, octanoic acid, palmitic acid, stearic acid, behenic acid, linoleic acid, linolenic acid, lipoic acid, oleic acid, isosteric acid, elaidoic acid, 2-ethylhexaneic acid, coconut oil fatty acid, tallow fatty acid, hardened tallow fatty acid, palm kernel fatty acid, lanolin fatty acid or similar acids. These may be substituted or unsubstituted. When substituted they are preferably substituted with hydroxyl, or sulphur containing groups such as but not limited to SO₃H, SH, or S—S.

In an embodiment of the current invention, the peptide is R₁—X—R₂.

R₁ and/or R₂ groups respectively bound to the amino-terminal (N-terminal) and carboxyl-terminal (C-terminal) of the peptide sequence.

In one embodiment, the peptide is R₁—X. Alternatively, the peptide is X—R₂.

Preferably, R₁ is H, C₁₋₄alkyl, acetyl, benzoyl or trifluoroacetyl;

X is the peptide of the invention or any modification mentioned;

R₂ is OH or NH₂.

In an embodiment, R1 is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, Tert-butyloxycarbonyl, 9-fluorenylmethyloxycarbonyl (Fmoc) and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl; R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not α-amino acids.

In accordance with another preferred embodiment, R₂ is —NR₃R₄, —OR₃ or —SR₃ wherein R₃ and R₄ are independently selected from the group formed by H, substituted or unsubstituted C₁-C₂₄ alkyl, substituted or unsubstituted C₂-C₂₄ alkenyl, Tert-butyloxycarbonyl, 9-fluorenylmethyloxycarbonyl (Fmoc), substituted or unsubstituted C₂-C₂₄ alkynyl, substituted or unsubstituted C₃-C₂₄ cycloalkyl, substituted or unsubstituted C₅-C₂₄ cycloalkenyl, substituted or unsubstituted C₃-C₂₄ cycloalkynyl, substituted or unsubstituted C₆-C₃₀ aryl, substituted or unsubstituted C₇-C₂₄ aralkyl, substituted or unsubstituted heterocyclyl ring of 3-10 members, and substituted or unsubstituted heteroarylalkyl of 2 to 24 carbon atoms and 1 to 3 atoms other than carbon wherein the alkyl chain is of 1 to 6 carbon atoms. Optionally, R₃ and R₄ can be bound by a saturated or unsaturated carbon-carbon bond, forming a cycle with the nitrogen atom. More preferably R₂ is —NR₃R₄ or —OR₃, wherein R₃ and R₄ are independently selected from the group formed by H, substituted or unsubstituted C₁-C₂₄ alkyl, substituted or unsubstituted C₂-C₂₄ alkenyl, substituted or unsubstituted C₂-C₂₄ alkynyl, substituted or unsubstituted C₃-C₁₀ cycloalkyl, substituted or unsubstituted C₆-C₁₅ aryl and substituted or unsubstituted heterocyclyl of 3-10 members, substituted or unsubstituted heteroarylalkyl with a ring of 3 to 10 members and an alkyl chain of 1 to 6 carbon atoms. More preferably R₃ and R₄ are selected from the group formed by H, methyl, ethyl, hexyl, dodecyl, or hexadecyl. Even more preferably R₃ is H and R₄ is selected from the group formed by H, methyl, ethyl, hexyl, dodecyl, or hexadecyl. In accordance with an even more preferred embodiment, R₂ is selected from —OH and —NH₂.

In accordance with another embodiment of this invention R₁ is selected from the group formed by H, acetyl, lauroyl, myristoyl or palmitoyl, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂.

In a preferred embodiment, the acyl (or acetyl) group is bound to the N-terminal end of at least one amino acid of the peptide.

In an embodiment of the invention, the peptide is modified to comprise a side chain protecting group. The side chain protecting group may be one or more of the group comprising benzyl or benzyl based groups, t-butyl-based groups, benzyloxy-carbonyl (Z) group, and allyloxycarbonyl (alloc) protecting group. The side chain protecting group may be derived from an achiral amino acid such as achiral glycine. The use of an achiral amino acid helps to stabilise the resultant peptide and also facilitate the facile synthesis route of the present invention. Preferably, the peptide further comprises a modified C-terminus, preferably an amidated C-terminus. The achiral residue may be alpha-aminoisobutyric acid (methylalaine). It will be appreciated that the specific side chain protecting groups used will depend on the sequence of the peptide and the type of N-terminal protecting group used.

In one embodiment of the invention the peptide is conjugated, linked or fused to one or more polyethylene glycol polymers or other compounds, such as molecular weight increasing compounds. The molecular weight increasing compound is any compound that will increase the molecular weight, typically by 10% to 90%, or 20% to 50% of the resulting conjugate and may have a molecular weight of between 200 and 20,000, preferably between 500 and 10,000. The molecular weight increasing compound may be PEG, any water-soluble (amphiphilic or hydrophilic) polymer moiety, homo or co-polymers of PEG, a monomethyl-substituted polymer of PEG (mPEG) and polyoxyethylene glycerol (POG), polyamino acids such as poly-lysine, poly-glutamic acid, poly-aspartic acid, particular those of L conformation, pharmacologically inactive proteins such as albumin, gelatin, a fatty acid, olysaccharide, a lipid amino acid and dextran. The polymer moiety may be straight chained or branched and it may have a molecular weight of 500 to 40.000 Da, 5000 to 10.000 Da, 10.000 to 5.000 Da. The compound may be any suitable cell penetrating compound, such as tat peptide, penetratin, pep-1. The compound may be an antibody molecule. The compound may be a lipophilic moiety or a polymeric moiety.

The lipophilic substituent and polymeric substituents are known in the art. The lipophilic substituent includes an acyl group, a sulphonyl group, an N atom, an O atom or an S atom which forms part of the ester, sulphonyl ester, thioester, amide or sulphonamide. The lipophilic moiety may include a hydrocarbon chain having 4 to 30 C atoms, preferably between 8 and 12 C atoms. It may be linear or branched, saturated or unsaturated. The hydrocarbon chain may be further substituted. It may be cycloalkane or heterocycloalkane.

The peptide may be modified at the N-terminal, C-terminal or both. The polymer or compound is preferably linked to an amino, carboxyl or thio group and may be linked by N-termini or C-termini of side chains of any amino acid residue. The polymer or compound may be conjugated to the side chain of any suitable residue.

The polymer or compound may be conjugated via a spacer. The spacer may be a natural or unnatural amino acid, succinic acid, lysyl, glutamyl, asparagyl, glycyl, beta-alanyl, gamma-amino butanoyl.

The polymer or compound may be conjugated via an ester, a sulphonyl ester, a thioester, an amide, a carbamate, a urea, a sulphonamide.

A person skilled in the art is aware of suitable means to prepare the described conjugate.

Peptides can be chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties.

The peptides of the invention may be subjected to one or more modifications for manipulating storage stability, pharmacokinetics, and/or any aspect of the bioactivity of the peptide, such as, e.g., potency, selectivity, and drug interaction. Chemical modification to which the peptides may be subjected includes, without limitation, the conjugation to a peptide of one or more of polyethylene glycol (PEG), monomethoxy-polyethylene glycol, dextran, poly-(N-vinyl pyrrolidone) polyethylene glycol, propylene glycol homopolymers, a polypropylene oxide/ethylene oxide co-polymer, polypropylene glycol, polyoxyethylated polyols (e.g., glycerol) and polyvinyl alcohol, colominic acids or other carbohydrate based polymers, polymers of amino acids, and biotin derivatives.

Modified peptides also can include sequences in which one or more residues are modified (i.e., by phosphorylation, sulfation, acylation, amindation, PEGylation, etc.), and mutants comprising one or more modified residues with respect to a parent sequence. Amino acid sequences may also be modified with a label capable of providing a detectable signal, either directly or indirectly, including, but not limited to, radioisotope, fluorescent, and enzyme labels. Fluorescent labels include, for example, Cy3, Cy5, Alexa, BODIPY, fluorescein (e.g., FluorX, DTAF, and FITC), rhodamine (e.g., TRITC), auramine, Texas Red, AMCA blue, and Lucifer Yellow. Preferred isotope labels include ³H, ¹⁴C, 32 P, ³⁵S, ³⁶Cl, ⁵¹Cr, ⁵⁷Co, ⁵⁸Co, ⁵⁹Fe, ⁹⁰Y, ¹²⁵I, ¹³¹I, and ²¹⁶Re. Preferred enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSA™), are known in the art, and are commercially available.

In an embodiment, the peptide, variant and/or composition is modified to increase drug performance ability. In an embodiment, the peptide, variant and/or composition is modified to increase stability, permeability, maintain potency, avoid toxicity and/or to increase half-life. The modification may be as described above. For example, the modification may be to protect the N and C-terminus, it may be a modified amino acid, cyclisation, replacement of an amino acid, and/or conjugation to macromolecules or large polymers or long life plasma proteins. Strategies to extend a half-life may be as described by Strohl et al. (BioDrugs, 2015), Schlapschy et al. (Protein Eng Des Sel. 2013), Podust et al. (Protein Eng Des Sel. 2013), Zhang, L et al. (Curr Med Chem. 2012), Gaberc-Porekar, V et al. (Curr Opin Drug Discov Devel. 2008). Examples include using PEGylation, lipidation (covalent binding of fatty acids to peptide side chains), fusion to Fc domains and human serum albumin, fusion with a hydrophilic amino acid polymer, e.g. XTEN or PAS, and/or fusion with half-life extension proteins.

Peptides or proteins can comprise weak sites in their sequence which are prone to undergoing proteolytic breakage when in a proteolytic enriched environment, e.g. in the blood or gastrointestinal tract. In an embodiment, the peptide, variant and/or composition comprises a modification of one or more weak sites such that the peptide, variant and/or composition does not undergo proteolytic breakdown/cleavage or undergoes a decreased amount of proteolytic breakdown/cleavage compared to an unmodified peptide or protein. Thus, the peptide may be modified to increase the resistance of the modified peptide to proteolytic degradation to mammalian gastrointertinal proteases.

Modification of peptides to extend the in vivo half-life of the peptide is described in the literature, for example:

Strategies to improve plasma half life time of peptide and protein drugs. Werle M, Bernkop-Schnürch A. (Amino Acids. 2006).

Modifications of the peptide/protein can lead to prolonged plasma half-life times

Strategies to Protect Peptides from Proteolysis

Many approaches are available to enhance stability of peptides through structure modification. Some approaches not only improve stability, but also enhance other ADME properties, e.g., cyclization can increase stability and permeability; conjugation to macromolecules can improve stability and reduce renal clearance. It is important to maintain potency and avoid toxicity while improving stability and ADME properties of peptides.

Protecting N- and C-Terminus

A number of proteolytic enzymes in blood/plasma, liver or kidney are exopeptidases, aminopeptidases and carboxypeptidases and they break down peptide sequences from the N- and C-termini. Modification of the N- or/and C-termini can often improve peptide stability. Many examples have reported that N-acetylation, and C-amidation increase resistance to proteolysis.

Replacing L-Amino Acids with D-Amino Acids

Substituting natural L-amino acids with nonnatural D-amino acids decreases the substrate recognition and binding affinity of proteolytic enzymes and increases stability. One example is vasopressin, which contains an L-Arg and has a half-life of 10-35 min in humans. The D-Arg analog, desmopressin, has a half-life of 3.7 h in healthy human volunteers. In the study of a bicyclic peptide inhibitor of the cancer-related protease urokinase-type plasminogen activator (uPA), replacement of a specific glycine with a D-serine not only improves potency by 1.8-fold but also increases stability by 4-fold in mouse plasma.

Modification of Amino Acids

Modification of natural amino acids can improve the stability of peptides by introducing steric hindrance or disrupting enzyme recognition. For example, gonadotropin-releasing hormone has a very short half-life (minutes), while buserelin, in which one Gly is replaced with a t-butyl-D-Ser and another Gly is substituted by ethylamide, has a much longer half-life in humans.

Cyclization

Cyclization introduces conformation constraint, reduces the flexibility of peptides, and increases stability and permeability. Depending on the functional groups, peptides can be cyclized head-to-tail, head/tail-to-side-chain, or side-chain-to-side-chain. Cyclization is commonly accomplished through lactamization, lactonization, and sulfide-based bridges. Disulfide bridges create folding and conformational constraints that can improve potency, selectivity, and stability. A number of disulfide bond-rich peptides are on the market or in preclinical or clinical development, e.g., linaclotide, lepirudin, and ziconotide.

Conjugation to Macromolecules

Conjugation to macromolecules (e.g., polyethylene glycol (PEG), albumin) is an effective strategy to improve stability of peptides and reduce renal clearance.

Renal Clearance

Many peptides exhibit promising in vitro pharmacological activity but fail to demonstrate in vivo efficacy due to very short in vivo half-life (minutes). The rapid clearance and short half-life of peptides hamper their development into successful drugs. The main causes of rapid clearance of peptides from systemic circulation are enzymatic proteolysis or/and renal clearance. The peptide may be modified such that renal clearance is reduced and a prolong half-life is noted.

These include the following:

Increase Plasma Protein Binding:

Renal clearance of peptides is reduced when they are bound to membrane proteins or serum proteins. An example is the cyclic peptide drug octreotide

Covalent Linkage to Albumin-Binding Small Molecules:

Covalently attaching albumin-binding small molecules to peptides can reduce glomerular filtration, improve proteolytic stability, and prolong half-life by indirectly interacting with albumin through the highly bound small molecules.

Conjugation to Large Polymers

Conjugation of peptides to large synthetic or natural polymers or carbohydrates can increase their molecular weight and hydrodynamic volume, thus reducing their renal clearance. The common polymers used for peptide conjugation are PEG, polysialic acid (PSA), and hydroxyethyl starch (HES).

Fusion to Long-Live Plasma Proteins

Covalent linkage of peptides to albumin or IgG fragments can reduce renal clearance and prolong half-life.

PEGylation

The attachment of long chains of the hydrophilic polymer polyethylene glycol to molecules of interest

Lipidation

A second major chemical modification method to increase peptide half-life is lipidation, which involves the covalent binding of fatty acids to peptide side chains.

PEGylation and lipidation both confer protection against proteases and peptidases by shielding through steric hindrance and extend circulating half-life through increased hydrodynamic radius, directly or indirectly.

Classical Genetic Fusions: Fc and HSA

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HAS

Designed Polypeptide Fusions: XTEN and PAS

The peptide may be provided as a fusion partners. For example using Fc, HAS, XTEN (Amunix) which is 864 amino acids long and comprised of six amino acids (A, E, G, P, S and T) or PAS (XL-Protein GmbH).

These methods enable the creation of molecules based on recombinant polypeptide-based partners that impart longer half-life.

Exemplary Dosages and Administration Strategies

As described above, the peptide(s) of the invention or composition(s) of the invention may include a “therapeutically effective amount” of a peptide of the invention. To better illustrate particular aspects, a detailed discussion of dosage principles is further provided here.

In practicing the invention, the amount or dosage range of the peptide of the invention employed typically is one that effectively induces, promotes, or enhances a physiological response associated with peptide of the invention binding to AT2. In one aspect, the dosage range is selected such that the peptide of the invention employed induces, promotes, or enhances a medially significant effect in a patient suffering from or being at substantial risk of developing a cardiovascular disease or a disease requiring neuro-regeneration, or pain.

In still another aspect, a daily dosage of active ingredient (e.g., peptide of the invention) of about 1 μg to 10 μg per per kilogram of body weight is provided to a patient. Ordinarily, about 1 to about 5 or about 1 to about 10 μg per kilogram per day given in divided doses of about 1 to about 6 times a day or in sustained release form may be effective to obtain desired results.

As a non-limiting example, treatment can be provided by administration of a daily dosage of peptide of the invention in an amount of about 1 μg to 10 μg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 μg/kg, per day, or 0.5, 0.9, 1.0, 1.1, 1.5, 2, 3, 4, 5, 6, 7, 8, 9, 10 μg/kg, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every or any combination thereof.

In another aspect, the invention provides the use of a peptide or composition of the invention (such as a combination composition) in the manufacture of a medicament used in the treatment of any of the foregoing conditions.

### EXAMPLES

When used in the Examples Ang-(1-6) has a sequence of SEQUENCE ID NO. 1.

When used in the Examples Ala¹-Ang-(1-6) has a sequence of SEQUENCE ID NO. 2.

When used in the Examples Ser⁴-Ang-(1-6) has a sequence of SEQUENCE ID NO. 3.

When used in the Examples Ala¹-Ser⁴-Ang-(1-6) has a sequence of SEQUENCE ID NO. 4.

When used in the Examples Ang-(2-6) has a sequence of SEQUENCE ID NO. 5.

When used in the Examples Ser⁴-Ang-(2-6) has a sequence of SEQUENCE ID NO. 6.

When used in the Examples Lys²-Ang-(1-6) has a sequence of SEQUENCE ID NO. 7.

When used in the Examples Ile³-Val⁵-Ang-(1-6) has a sequence of SEQUENCE ID NO. 8.

When used in the accompanying Figures, the term EA, refers to Ang (1-6).

Materials and Methods

Cell Culture Conditions, Transfection and Stimulation

Human embryonic kidney (HEK-293) cells were cultured in DMEM medium supplemented with FBS (10%), HEPES buffer (1%), sodium pyruvate (1%), and L-glutamine (1%) and maintained under standard conditions (5% CO₂, 95% humidity and 37° C.). Cells were cultured in 100 mm cell culture dishes and seeded in 48-well plates at a density of 75,000 cells per well. The next day, HEK-293 cells were transfected using a transient transfection procedure following the manufacturer's instructions. Briefly, 150 ng of control plasmid pcDNA3.1 or a combination of 50 ng of pcDNA3.1 and 100 ng of expression vectors containing the cDNA for Mas, MrgD, AT1 or AT2, were mixed with serum-free medium and PolyFect transfection reagent. After 10 min incubation at room temperature, which allowed the complex formation, complete medium was added and the total volume was transferred into appropriate wells of the 48-well plate. The cells were incubated for 16-20 hrs. The next day, the medium was replaced by serum-free medium 1 hr before stimulation. After stimulation with A779, D-Pro, PD123319, forskolin (all 10⁻⁶ M), or PTX (50 ng/ml), or SQ22536 (2×10⁻⁶M) for 15 min, the solvent, Ang-(1-6) or C21 (all in the mentioned concentrations) were added for 15 min. Then, the cells were lysed by adding 180 μl/well of 0.1M hydrochloric acid with 0.1% Triton X-100, and the lysates were stored at −80° C. until cAMP measurement. The protein concentration was determined using Pierce BCA Protein Assay Kit according to the manufacturer's protocol (Thermo Fisher Scientific, Waltham, Mass., USA).

Isolation and Culture of Primary Cells and their Stimulation

Kidney mesangial cells (MC) were isolated from 10-12-week old mice deficient in AT2, and from their age- and gender-matched C57/BL6 control, according to the protocol previously described (Zhu et al., Mol Cell Endocrinol. 2013). MC were cultured in 75 cm² tissue culture flasks and seeded at passage 2 for stimulation in 24-well plates at a density of 100,000 cells per well.

Primary human umbilical vein endothelial cells (HUVEC) were purchased from PromoCell (Heidelberg, Germany). HUVEC were cultured in 100 mm cell culture dishes and were seeded at 75,000 per well in 24-well plates at passage 5-7, for stimulation. Stimulation with Ang-(1-6) or C21 and the blockers was carried out as described above.

Measurement of cAMP in Cell Lysates

cAMP concentration in cell lysates was determined using Direct cAMP ELISA kit (Enzo Life Sciences Ltd., Exeter, United Kingdom). Briefly, wells of 96-well plate (Goat Anti-Rabbit IgG pre-coated) were neutralized with 50 μl of Neutralizing Reagent. Next, 100 μl of acetylated cAMP standard or cell lysate was added, followed by 50 μl of blue cAMP-Alkaline Phosphatase Conjugate and 50 μl of yellow EIA Rabbit Anti-cAMP antibody. The plate was then incubated on a shaker (˜400 rpm) at room temperature for 2 h. Next, the wells were aspirated and rinsed three times with Wash Buffer (1:10, Tris buffered saline containing detergents and sodium azide in deionized water). After the final wash, the plate was tapped against clean paper towel to remove any remaining Wash Buffer. To each well, 200 μl p-Nitrophenyl Phosphate Substrate Solution was added, and the plate was incubated for 1 h at room temperature. The enzymatic reaction was stopped by adding 50 μl of Stop Solution, and the absorbance at 405 nm was measured immediately. The cAMP concentration was determined from non-linear standard curve using GraphPad Prism 6.0 software.

Dual Luciferase Assay (DLR)

For Dual-Luciferase reporter assays, HEK293 cells were seeded into 48-well plates (75,000 cells/well). About 24 h later, cells were transiently transfected with 100 ng eukaryotic expression vectors of AT1 or AT2 together with 25 ng pELK-Luc, pNFAT-Luc luciferase or pCREB-Luc Reporter Vectors (Signosis, Santa Clara, Calif., USA) and 25 ng pRL-TK (Promega GmbH, Mannheim, Germany) as previously described. Next day, the medium was replaced by serum-free medium 1 h before stimulation. After 6 hours of stimulation with C21 (10⁻⁶ M to 10⁻¹⁴ M), Ang-(1-6) (10⁻⁶ M to 10⁻¹⁴ M) or Ang II (10⁻⁶M to 10⁻⁷M), cells were lysed with 70 μL/well passive lysis buffer provided with the Dual Luciferase Reporter Assay kit (Promega GmbH, Mannheim, Germany) and incubated on a shaker for 15 min at room temperature. The activity for Firefly and Renilla Luciferase was measured with Orion L Microplate Luminometer (Berthold Detection Systems GmbH, Pforzheim, Germany) according to the manufactures protocol. Briefly, 30 μL of lysed cells where transferred into a white 96-MicroWell plate (Thermo Fisher Scientific, Waltham, Mass., USA), and 100 μL of Luciferase Assay Reagent II was added. After quantifying the firefly luminescence, the reaction was quenched by adding 100 μL of Stop&Glo reagent and renilla activity was measured. For calculations, the ratio of firefly and renilla luciferase was used.

Peptide Degradation

Peptides (10⁻⁴M) were incubated with 45 μg of murine kidney membrane preparations and diluted with 50 mM Tris-HCl buffer pH 7.4 laced with 0.1% BSA to minimise adhesion of the peptide onto the tube walls. Incubations proceeded for the desired time, after which the reaction was stopped by addition of half volume of 350 mM HClO₄. Samples were then centrifuged to remove any particulates prior to LCMS analysis.

In Silico Modelling

Docking of Ang-(1-6) was performed with Glide version 74011 (Schrödinger LCC, New York, N.Y.). The receptor AT2 was prepared with the protein preparation wizard module in Maestro (Schrödinger LCC, New York, N.Y.) keeping only the protein, the ligand and water molecules and setting the pH to 7.4±2 (ligand final state had a deprotonated tetrazole group). Protein was superposed to the modelled Mas receptor and grid box was built equivalently plus switching the option for peptide docking on. Ang-(1-6) was prepared from Ang-(1-7) (removing last residue of the latter) and generating two neutral ionisation states for the histidine residue.

Endothelial Barrier Study

Human Retinal Microvascular Endothelial Cells (HRMECs) were seeded at a density of 20,000 cells per well. Cells were left to form a barrier 23 hours. When the curve showed a plateau, plates were taken from the machine and cells were treated for 15 minutes with drug concentrations (diagram A). Drugs were added to medium in each well in order to avoid disruption of barrier or detachment of cells. After treatments media was replaced with new media containing VEGF (50 ng/ml) or PBS (Vehicle). Plates were then transferred into the machine and experiment started again (diagram B). Once finished, the xCelligence data was exported to Microsoft Excel, where the data from the drug response was transferred to Prism for analysis. Data was normalised to the time-point when VEGF was added. Time point for analysis was chosen based on previous optimisation and experiments with the same cell type (effect of VEGF after 15-30 minutes after addition).

Epithelial Barrier Study

Intestinal preparations were mounted in Ussing chambers (exposed area of 0.12 cm²) with 5 ml of Krebs solution (95% O2/5% CO2, 37° C.) in the basolateral and luminal reservoirs. Tissues were voltage-clamped at 0 mV using an automatic voltage clamp (EVC 4000, World Precision Instruments, Sarasota, Fla.) and the short-circuit current (Isc) required to maintain the potential at 0 mV was monitored as a reflection of the net active ion transport across the epithelium. 0.1 μg PTX (Pertussis toxin) was used to compromise barrier function, and after 10 minutes Ang-(1-6) and peptide modifications were added to the chambers in a 10⁻¹¹ M concentration. Resistance was calculated using Ohms law. Experiments were carried out simultaneously in chambers connected to a PC equipped with DataTrax II software (WPI).

Colony Formation Assay

Human kidney cancer A498 cells were seeded at 300 cells/well in a 6-well plate. The next day, cells were treated with various peptides daily. Each condition was run in triplicate. After 5 days of treatment, cells were washed once with PBS after media removal, stained with 0.5% crystal violet in 30% ethanol and 3% formaldehyde for 10 min at RT and washed with distilled water. Colonies were counted under the microscope.

Vascular Reactivity Experiments in Mesenteric Microvessels

Mice were anaesthetized with 70 mg kg-1 I.P. sodium pentobarbital and exsanguinated. Third-branch mesenteric arteries (mean internal diameter ranging between 150 and 200 μm) were mounted as ring preparations on a small-vessel myograph to measure isometric tension as described before (Vallejo et al. 2000; Peiro' et al. 2007). Arteries were contracted with 10 μmol I-1 noradrenaline (NA; Sigma, St Louis, Mo., USA), and then the vasoactive response to Ang-(1-6) and C21 (Bachem, Bubendorf, Switzerland; 1 μmol I-1 to 1 μmol l-1) were tested by adding increasing concentrations of the drug.

### Example 1

Effect of Ang (1-6) on cAMP in AT2-Transfected HEK293 Cells.

Methodology Summary

Ang (1-7) has a sequence of DRVYIHP, not being able to stimulate the AT2 receptor, but Mas and MrgD.

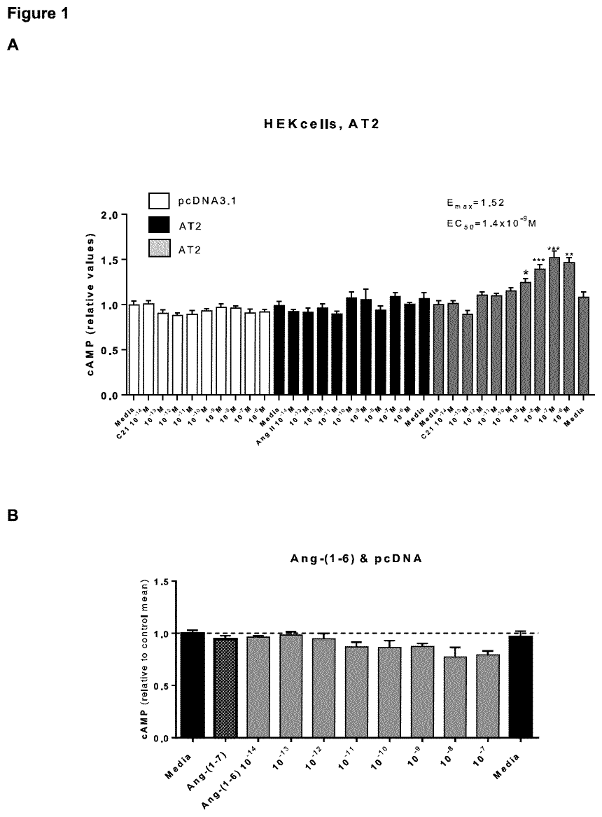

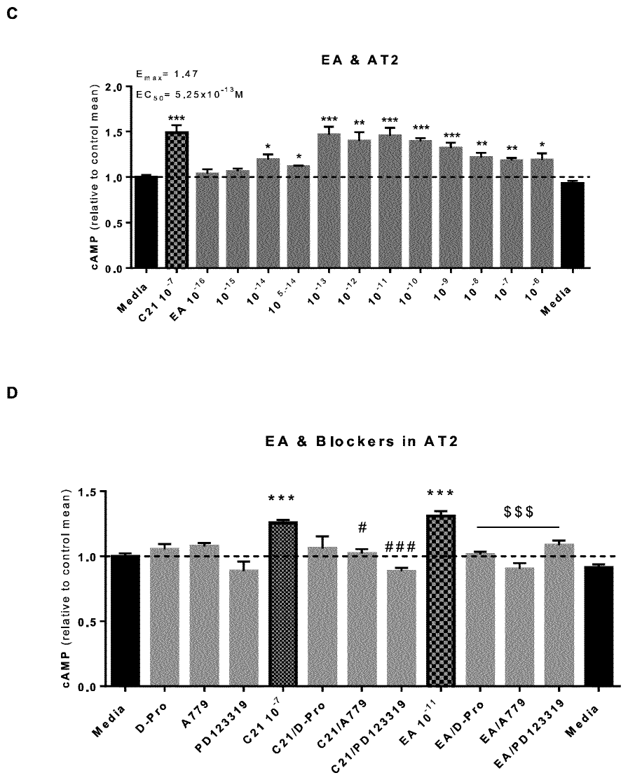

AT2-transfected HEK293 cells were stimulated for 15 min with a range of concentrations (10⁻¹⁴ to 10⁻⁵ M) of C21, Ang II or PBS and the concentration of cAMP was measured in accordance with the methodology provided. The results are illustrated by FIG. 1A.

pcDNA-transfected HEK293 cells and AT2-transfected HEK293 cells were stimulated for 15 min with a range of concentrations (10⁻¹⁶ to 10⁻⁶ M) of Ang-(1-6). The results are illustrated by FIGS. 1B and 1C.

AT2-transfected HEK293 were stimulated for 15 min with blockers (A779, D-Pro⁷-Ang-(1-7) (D-Pro) and PD123319 (all 10⁻⁶ M)), followed by 15 min stimulation with Ang-(1-6) (EA) (10⁻¹¹M) or C21 (10⁻⁷M), an unspecific AT2 agonist. The results are illustrated by FIG. 1D.

Mas transfected HEK293 cells and MrgD-transfected HEK293 were stimulated for 15 min with a range of concentrations (10⁻¹⁴ to 10⁻⁷ M) of Ang-(1-6). The results are illustrated by FIGS. 1E and F. Ang-(1-7) acted as a positive control.

Results

Ang (1-6) stimulation increases intracellular cAMP production in AT2 transfected cells.

Ang (1-6) is not cross-reacting with the AT2 like receptors Mas and MrgD.

Results are expressed as mean±SEM. Data was reported as a fold change or percentage of the untreated control mean. ***P<0.001, significantly different from control; ^{###}P<0.001, ^{##}P<0.01 or ^{#}P<0.05, significantly different from C21 or Ang-(1-6); ANOVA with Bonferroni post-hoc test.

Conclusion

Ang (1-6) is an agonist of AT2.

### Example 2

Signalling Mechanisms of Ang-(1-6) and Effect of Ang-(1-6) in HUVEC and Mesangial Cells.

Methodology Summary

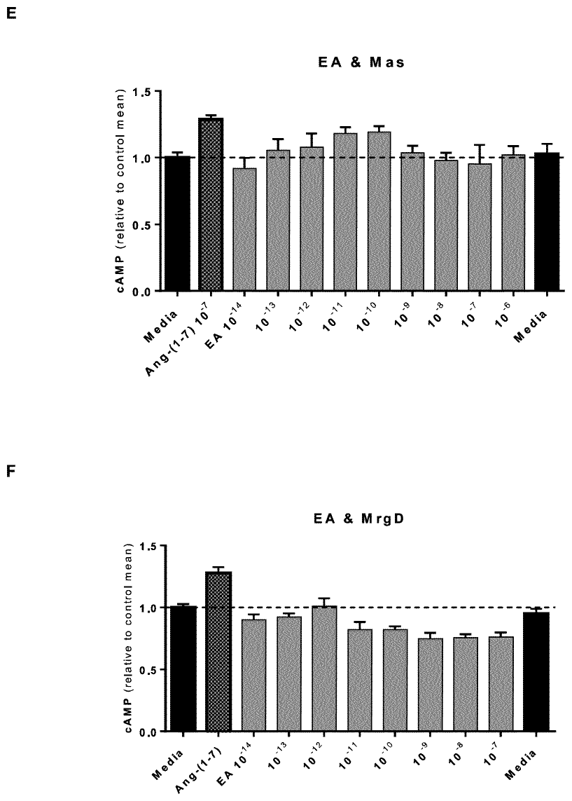

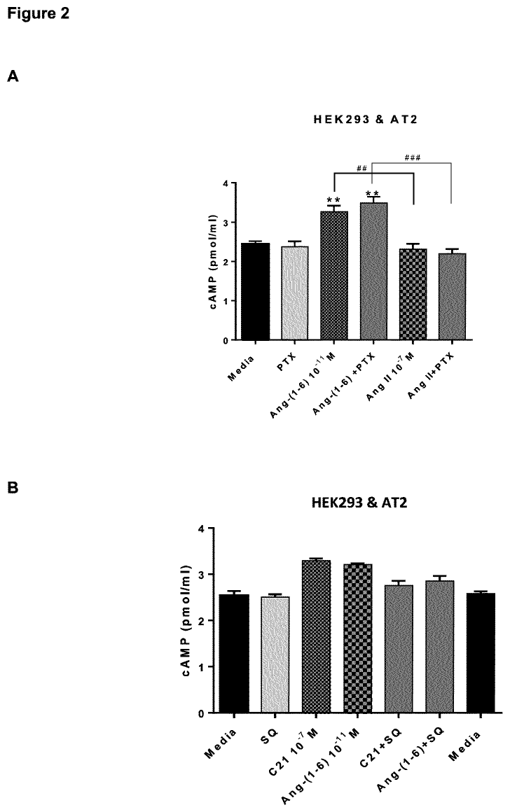

AT2-transfected HEK293 cells were stimulated for 6 h with PTX (50 ng/ml), an inhibitor of G_{alpha i} activation, followed by stimulation with Ang-(1-6) (10⁻¹¹ M) for a further 15 mins. The results are illustrated in FIG. 2A.

AT2-transfected HEK293 cells were stimulated for 15 min with SQ-22536 (2×10⁻⁶ M), an adenylyl cyclase inhibitor, followed by 15 min stimulation with Ang-(1-6) (10⁻¹¹ M). The results are illustrated in FIG. 2B.

HUVEC cells were stimulated for 15 min with a range of concentrations (10⁻¹⁴ to 10⁻⁶ M) of Ang-(1-6) or C21 before analysis of cAMP concentration. The results are illustrated in FIGS. 2C and D.

WT or AT2 KO Primary mesangial cells were stimulated for 15 min with a range of concentrations (10⁻⁷ to 10⁻¹³ M) of Ang-(1-6), or cells were pre-stimulated for 15 min with blockers (A779, D-Pro⁷-Ang-(1-7) (D-Pro) and PD123319 (all 10⁻⁶ M)), followed by 15 min stimulation with Ang-(1-6). The results are illustrated in FIG. 2E to 2G.

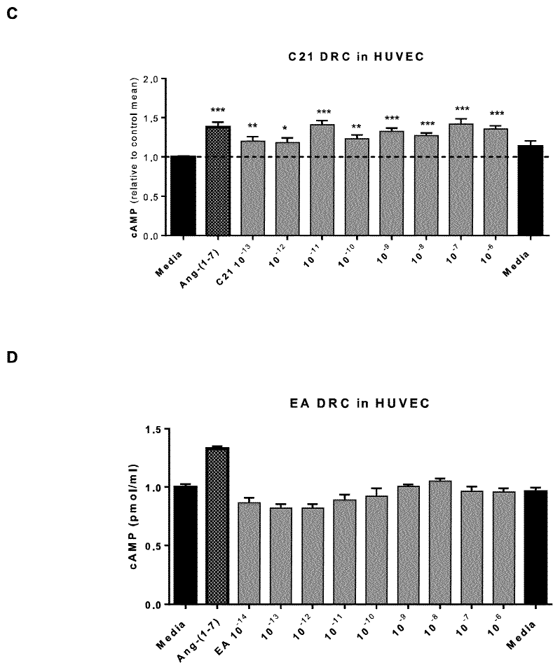

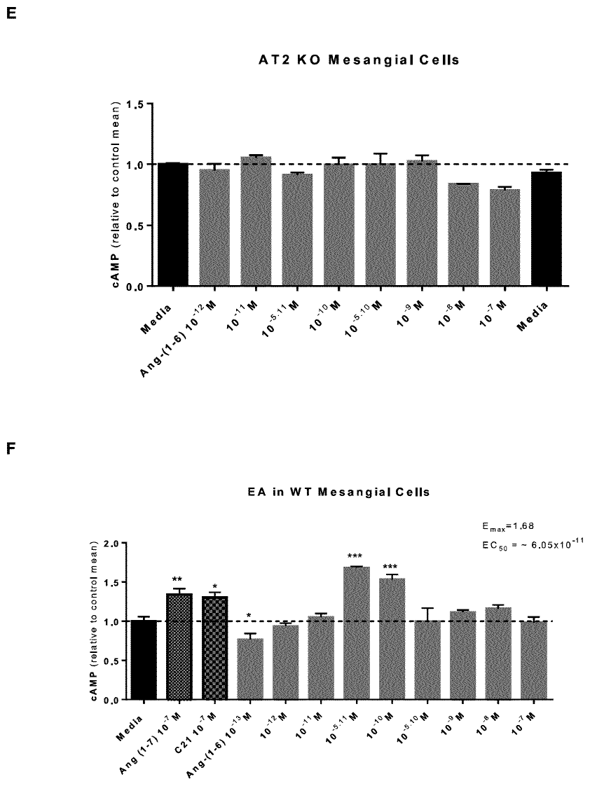

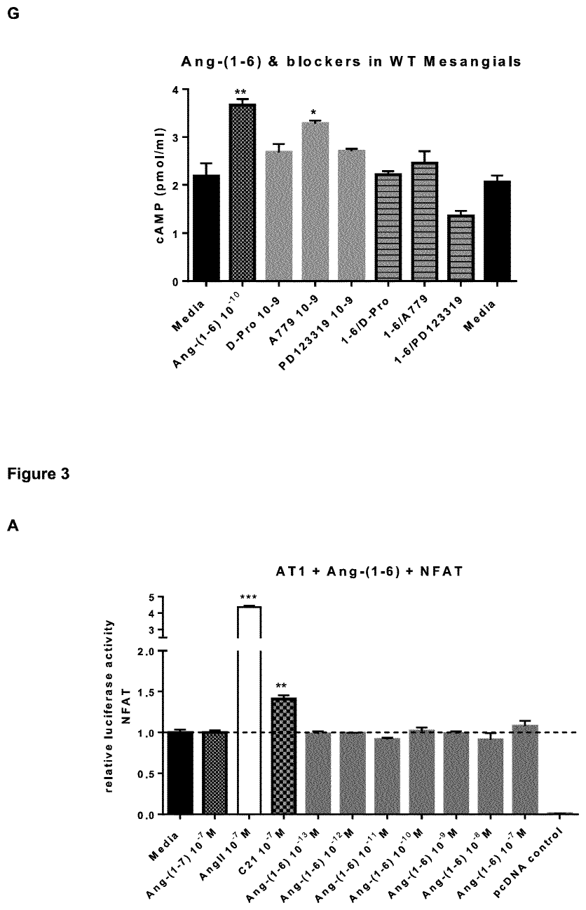

HEK293 cells were transiently co-transfected with pcDNA3.1, AT2, orAT1, and pNFAT-Luc, pCREB-Luc or pElk1-Luc. The cells were stimulated with PBS, C21, Ang II or Ang-(1-6). Luciferase production was measured. The results are illustrated in FIG. 3A to 3C.

Results

Results are expressed as mean±SEM. Data was reported as a fold change or percentage of the untreated control mean. ***P<0.001, significantly different from the MrgD control; ^{###}P<0.001, significantly different from Ang-(1-6) or C21; ANOVA with Bonferroni post-hoc test.

Conclusion

Ang (1-6) is the agonist for the AT2 receptor, increasing cAMP by activating adenylyl cyclase. Receptor/agonist interaction leads to G_{alpha s} but not G_{alpha i} activation Ang-(1-6) stimulates cAMP in primary kidney cells and this stimulation is completely AT2-dependent. Ang-(1-6) cannot stimulate primary endothelial cells, because they do not express AT2. C21 indeed can stimulate such cells, since it can also stimulate Mas and MrgD, which are expressed in HUVEC.

### Example 3

Ang-(1-6) Peptide Degradation, in Silico Modelling, and Displacement Studies.

Methodology Summary

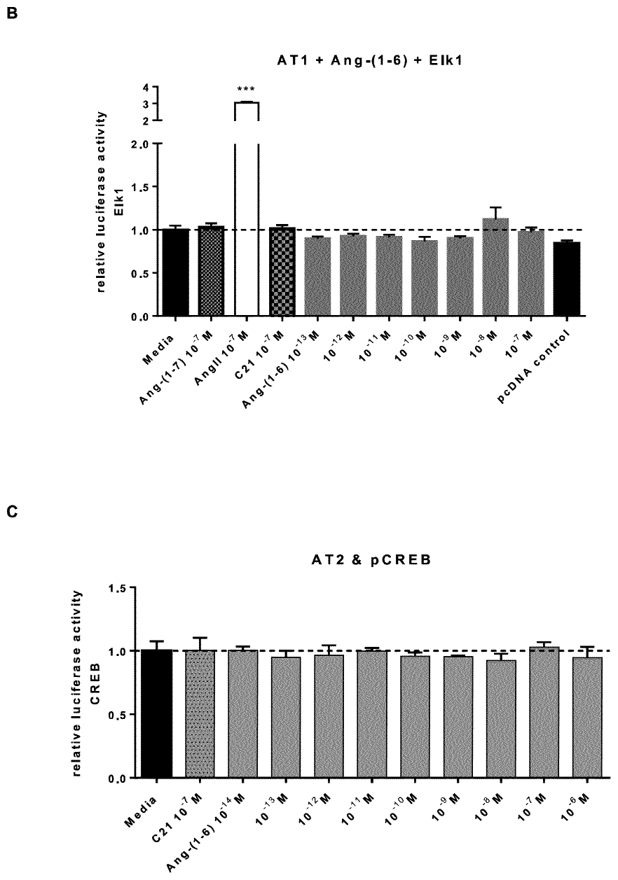

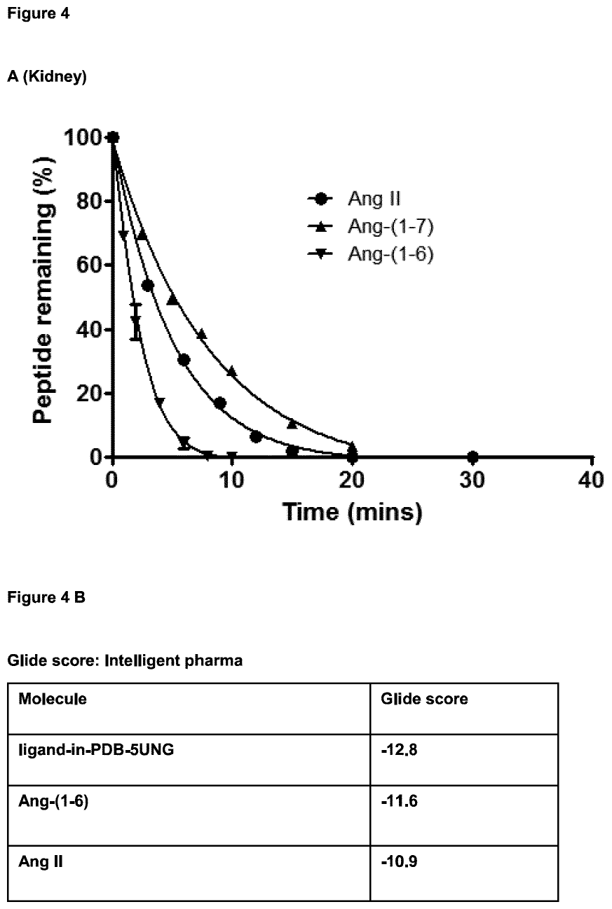

Ang-(1-6) degradation in mouse kidney membranes over 30 minutes compared to Ang-(1-7) and Ang II. (FIG. 4A)

Ang-(1-6), Ang II and the PDB 5UNG ligand were docked into the binding site of the AT2 receptor. Gliding scores of the ligands into the AT2 receptor are listed in the FIG. 4B.

Conclusion

The in silico modelling confirms that Ang-(1-6) perfectly fits into the AT2 receptor, while Ang II does not.

### Example 4

Ang-(1-6) Effect on Epithelial and Endothelial Cell Function.

Methodology Summary

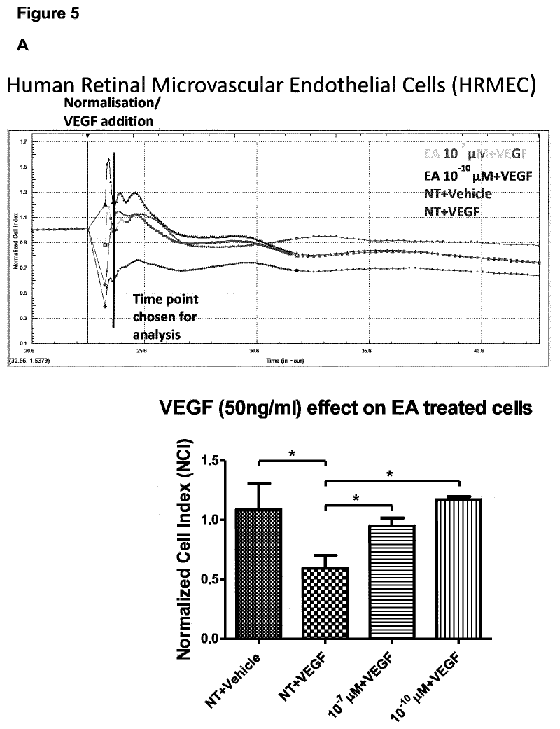

HRMEC cells were treated with Ang-(1-6) or PBS (vehicle) for 15 mins. This experiment was repeated with new media containing VEGF (50 ng/ml) or PBS (vehicle). xCelligence data was exported to Prism for analysis (left and right). (FIG. 5A)

Mesenteric arteries were contracted with NA (1 μmol/L). Artery relaxation was assessed by adding cumulative concentrations of Ang-(1-6) and C21 at 2 min intervals (final bath concentrations 10⁻¹⁴ to 10⁻⁵M), solvent (PBS) acted as control (FIG. 5B). Intestinal preparations were mounted in Ussing chambers. Pertussis toxin (toxin) was used to compromise barrier function, and Ang-(1-6) and peptide modifications were added to test for their effect on restoring barrier function (FIG. 5C). Results are expressed as mean±SEM. *P<0.05.; ANOVA with Bonferroni post-hoc test.

Conclusion

Ang (1-6) can protect the endothelial and epithelial barrier function, which is key in the diseases of the invention. Furthermore, Ang-(1-6) analogues with amino acid modification on amino acid 2 or 3 and 5 also show biological activity towards the protection of barrier function.

### Example 5

Effect of Ang (1-4) and Ang (1-7) on cAMP in AT2-Transfected HEK293 Cells.

Methodology Summary

Ang (1-4) has a sequence of DRVY

Ang (1-7) has a sequence of DRVYIHP

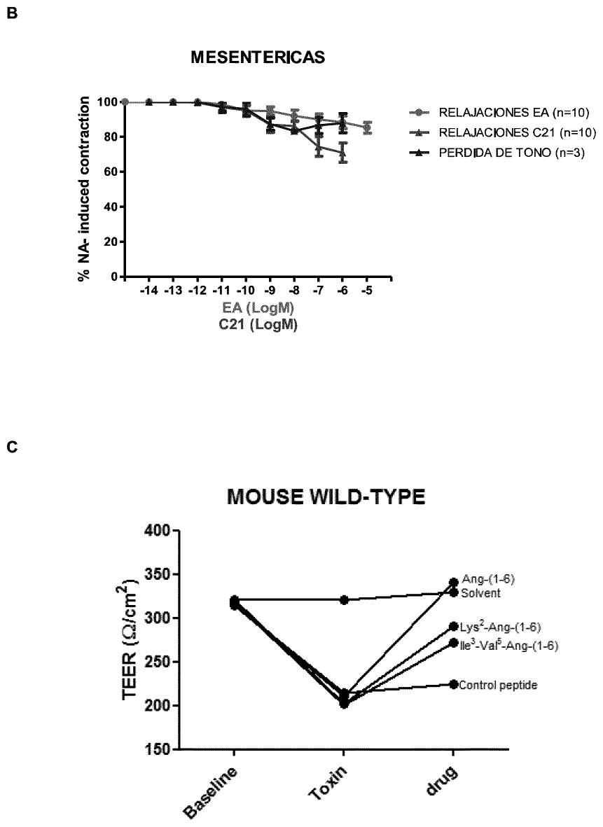

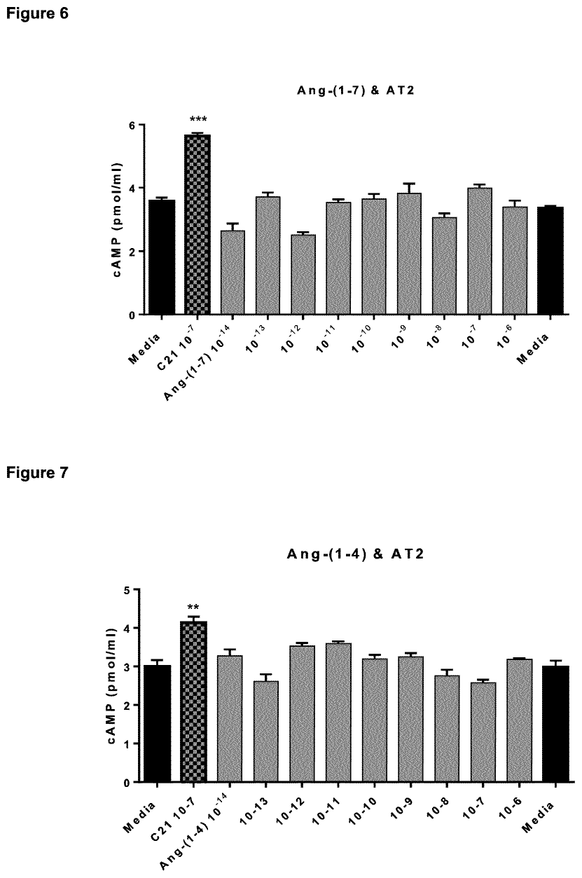

AT2-transfected HEK293 cells were stimulated for 15 min with a range of concentrations (10⁻¹³ to 10⁻⁶ M) of C21, Ang (1-4) and Ang (1-7) PBS and the concentration of cAMP was measured in accordance with the methodology provided. The results are illustrated by FIGS. 6 and 7.

Conclusion

Ang (1-4) and (Ang 1-7) do not increase intracellular cAMP. Ang (1-4) and Ang (1-7) are not agonists of AT2.

### Example 6

Effect of Ala¹-Ang-(1-6) Alone and on Ang (1-6) Mediated cAMP Signal in AT2-Transfected HEK Cells

Methodology Summary

Ala¹-Ang-(1-6) has the following sequence: ARVYIH

Cells were stimulated for 15 min with (a) 10⁻¹¹ M and 10⁻¹° M Ang-(1-6), (b) Ala¹-Ang-(1-6) (10⁻¹⁰ M) or (c) a range of concentrations (10⁻¹²M to 10⁻⁸M) of Ala¹-Ang (1-6) together with Ang-(1-6) at a concentration of 10⁻¹⁰M. The concentration of cAMP was measured in accordance with the methodology provided.

Results

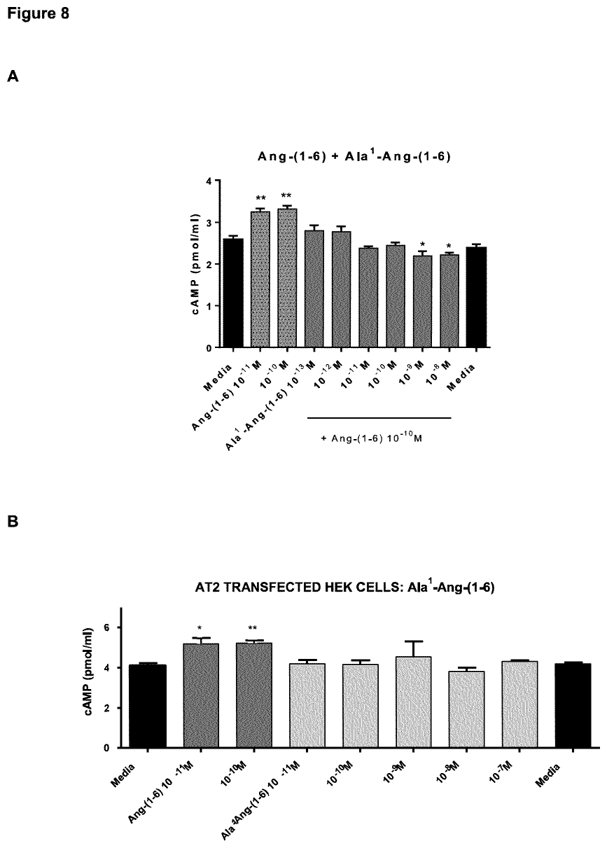

The cAMP-generating capacity of Ang-(1-6) (10⁻¹⁰) was completely blocked with increasing concentrations of Ala¹-Ang-(1-6). The results are illustrated in FIG. 8A-C.

Conclusion

Increasing concentrations of Ala¹-Ang-(1-6) block the biological effect of Ang-(1-6). Thus, he substitution of amino acid at position 1 from Arg to Ala transforms the Ang (1-6) agonist to an antagonist.

### Example 7

Effect of Ala¹-Ser⁴-Ang-(1-6) and Ang (1-6) on cAMP in AT2-Transfected HEK293 Cells

Methodology Summary

Ala¹-Ser⁴-Ang-(1-6) has a sequence of ARVSIH

AT2-transfected HEK293 cells were stimulated for 15 min with C21, Ang-(1-6) and a range of concentrations (10⁻¹⁴ to 10⁻⁷ M) of Ala¹-Ser⁴-Ang-(1-6), and the concentration of cAMP was measured in accordance with the methodology provided.

Results

Ang (1-6) and Ala¹-Ser⁴-Ang-(1-6) stimulation increases intracellular cAMP production. At the same concentration (10⁻¹¹M), there was a greater increase in intracellular cAMP production seen with Ala¹-Ser⁴-Ang-(1-6) compared with Ang (1-6).

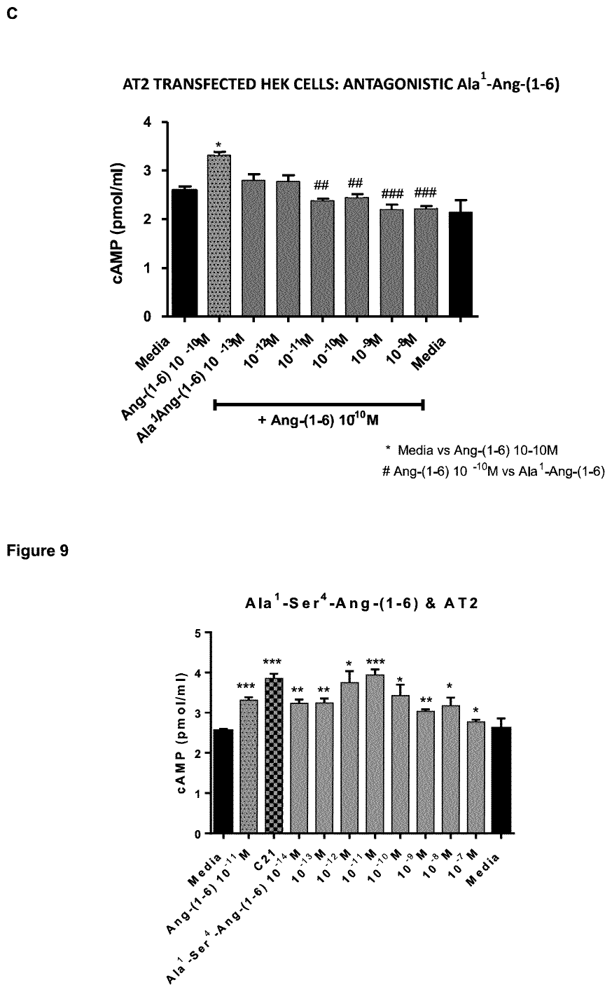

The results are illustrated by FIG. 9.

Conclusion

The substitution on amino acid 1 and 4 generates a very efficient agonist with Emax at 10⁻¹¹M. At the same concentration (10⁻¹¹M), there was a greater increase in intracellular cAMP production seen with Ala¹-Ser⁴-Ang-(1-6) compared with Ang (1-6). Thus, the exchange of two amino acids generates an AT2 agonist with own pharmacodynamics towards the receptor.

### Example 8

Effect of Ser⁴-Ang-(1-6) on cAMP in AT2-Transfected HEK293 Cells

Methodology Summary

Ser⁴-Ang-(1-6) has the following sequence: DRVSIH

AT2-transfected HEK293 cells were stimulated for 15 min with Ang-(1-6) and a range of concentrations (10⁻¹⁵ to 10⁻⁷ M) of Ser⁴-Ang-(1-6) and the concentration of cAMP was measured in accordance with the methodology provided.

Results

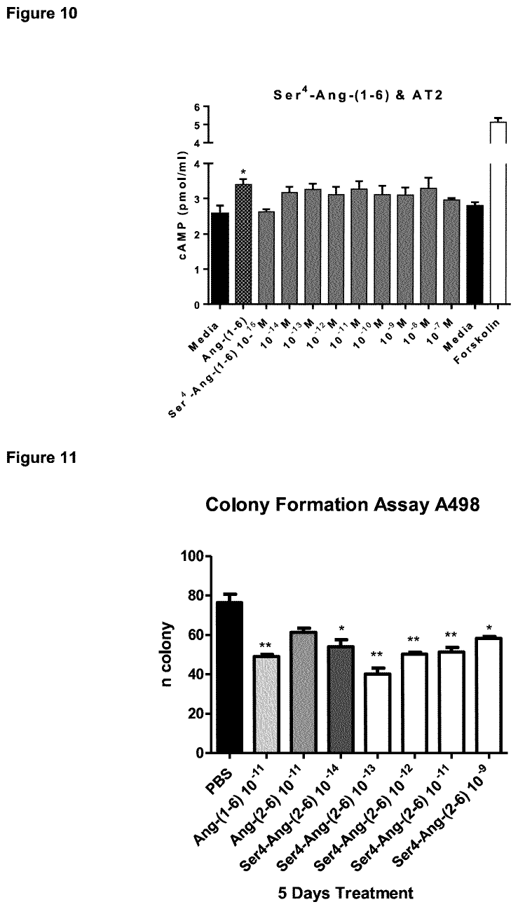

Ang (1-6) and Ser⁴-Ang-(1-6) stimulation increases intracellular cAMP production. The results are illustrated by FIG. 10.

Conclusion

Ser⁴-Ang-(1-6) is another modification of Sequence 1 that stimulates the AT2 comparable to Ang (1-6).

### Example 9

Ang (1-6) and analogues reduce the number of colonies of cancer cells.

Human renal cancer cells were treated with Ang-(1-6) or analogues and the effect on colony formation counted. Such cells are used for in vivo experiments in rodents when implanted subcutaneously or into the kidney to generate a rapidly forming and growing tumour, being leathal if untreated.

Results The results of a colony formation assay using Ang (1-6) (10⁻¹¹M), and Ang (2-6) (10⁻¹¹M), and Ser⁴-Ang (2-6) (10⁻¹⁴ to 10⁻⁹ M) illustrates a significant reduction of colonies in comparison to solvent (PBS)-treated cells (FIG. 11).

Conclusion:

An increase in colonies is a measure of more proliferation, migration and metastasis. Therefore, Ang (1-6) and its truncated version Ang (2-6) and Ser⁴-Ang (2-6) show anti-cancer properties.

### Example 10

In Vivo Rodent Model

Methodology

The human renal cells of EXAMPLE 9 may be used for in vivo experiments in rodent models. When implanted subcutaneously or into the kidney, the cells generate a rapidly forming and growing tumour, being lethal if untreated. The rodents may be subsequently treated, e.g. subcutaneously injected, with an effective or suitable amount of any one of the peptide of the invention in any suitable form. Tumour growth and/or development may be monitored over time. The peptide may be administered alone or in combination with an active agent, such as an anti-cancer agent.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A peptide comprising an amino acid sequence of SEQUENCE ID NO. 1, in which the sequence has 1, 2 or 3 amino acid changes.

2. The peptide of claim 1, in which the peptide comprises a maximum of 6 amino acids.

3. The peptide of claim 1, in which the peptide comprises 2 amino acid changes.

4. The peptide of claim 3, in which the peptide comprises an amino acid change at position 1 and an amino acid change at position 4.

5. The peptide of claim 3, in which the peptide comprises an amino acid change at position 3 and an amino acid change at position 5.

6. The peptide of claim 1, in which the peptide comprises 1 amino acid change.

7. The peptide of claim 6, in which the peptide comprises an amino acid change at position 1 or at position 4.

8. The peptide of claim 6, in which the peptide comprises an amino acid change at position 2.

9. The peptide of claim 1, in which the peptide comprises an amino acid sequence of any one of SEQUENCE ID NO. 2 to 8.

10. The peptide of claim 9, in which the peptide comprises an amino acid sequence of SEQUENCE ID NO. 4.

11. The peptide of claim 9, in which the peptide consists essentially of a sequence of any one of SEQUENCE ID NO. 2 to 8.

12. The peptide of claim 1, in which the peptide is a modified peptide.

13. The peptide of claim 12, in which the peptide is modified at the N-terminus and/or C-terminus.

14. The peptide of claim 1, in which the amino acid change is a substitution.

15. A composition comprising a therapeutically effective amount of a peptide of claim 1, or a peptide consisting of SEQUENCE ID NO. 1.

16-18. (canceled)

19. An antagonist of the AT2 receptor comprising a sequence of SEQUENCE ID NO. 2, or a functional variant or fragment thereof.

20.-28. (canceled)

29. A method of treating a disease, the method comprising administering a peptide of claim 1 or a peptide consisting of SEQ ID NO: 1.


SEQUENCE LISTING (8 sequences)

SEQ ID NO: 1 - protein, 6 aa, Artificial Sequence
  SYNTHETIC PEPTIDE
DRVYIH

SEQ ID NO: 2 - protein, 6 aa, Artificial Sequence
  SYNTHETIC PEPTIDE
ARVYIH

SEQ ID NO: 3 - protein, 6 aa, Artificial Sequence
  SYNTHETIC PEPTIDE
DRVSIH

SEQ ID NO: 4 - protein, 6 aa, Artificial Sequence
  SYNTHETIC PEPTIDE
ARVSIH

SEQ ID NO: 5 - protein, 5 aa, Artificial Sequence
  SYNTHETIC PEPTIDE
RVYIH

SEQ ID NO: 6 - protein, 5 aa, Artificial Sequence
  SYNTHETIC PEPTIDE
RVSIH

SEQ ID NO: 7 - protein, 6 aa, Artificial Sequence
  SYNTHETIC PEPTIDE
DKVYIH

SEQ ID NO: 8 - protein, 6 aa, Artificial Sequence
  SYNTHETIC PEPTIDE
DRIYVH